(19) United States
(12) Patent Application Publication
(10) Pub. No.: US 2009/0156509 A1
(43) Pub. Date: Jun. 18, 2009

(54) Tri-peptide Inhibitors of Serine Elastases

(75) Inventors: John C. Cheronis, Conifer, CO (US); Val Goodfellow, Encinitas, CA (US); Colin J. Loweth, San Marcos, CA (US); Shamsi Raeissi, San Diego, CA (US)

Correspondence Address: MERCHANT & GOULD PC, P.O. BOX 2903, MINNEAPOLIS, MN, 55402-0903, US

(73) Assignee: Accuthera, Inc., Conifer, CO (US)

(21) Appl. No.: 12/326,612

(22) Filed: Dec. 2, 2008

Related U.S. Application Data
(60) Provisional application No. 61/013,279, filed on Dec. 12, 2007.
(60) Provisional application No. 61/058,085, filed on Jun. 2, 2008.

(51) Int. Cl. A61K 38/06; C07K 5/097

(52) U.S. Cl. 514/18; 530/331

(57) ABSTRACT

The present invention provides compounds of formula (I):

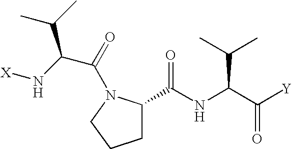

where X is R₁—(CR₃R₄)ₙOC(O)—; R₁—(CR₃R₄)ₙC(O)—; R₁—C(O)NH(CR₃R₄)ₙOC(O)—; R₁—C(O)NH(CR₃R₄)ₙC(O)—; R₁—C(O)(CR₃R₄)ₙOC(O)—; or R₁—C(O)(CR₃R₄)ₙC(O)—;
where R₁ is optionally substituted C₅₋₁₀ aryl or heteroaryl; OH or NH₂; where R₃ and R₄ are independently H or methyl; and
n is 0 to 6; and
Y is —CF₃ or one of:

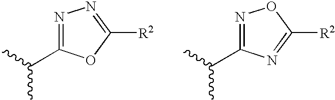

where R₂ is C₁₋₈ alkyl optionally substituted with halo or —OH; —(CR₆R₇)ₚ—C₅₋₆ aryl optionally substituted with halo, —OH, C₁₋₈ alkyl, C₁₋₈ haloalkyl, —(CH₂)ₘC(O)NH₂ or —(CH₂)ₘOCH₃;
where R₆ and R₇ are independently H or methyl; m is 0 to 4, and p is 0 or 1 or a pharmaceutically acceptable salt, ester, metabolite or prodrug thereof

### CROSS REFERENCE TO RELATED APPLICATIONS

This application claims the benefit of U.S. Provisional Application No. 61/013279, filed Dec. 12, 2007, and U.S. Provisional Application No. 61/058085, filed Jun. 2, 2008, the entire contents of each of which are hereby incorporated herein by reference.

### BACKGROUND OF THE INVENTION

Elastase is a general term that describes a group of enzymes (proteases or proteinases) that have the ability to degrade elastin. Elastin is the primary extracellular matrix protein that confers elastic qualities to a variety of tissues including the lung, skin and blood vessels. Different proteases from the serine, cysteine and metallo classes have been shown to degrade elastin with varying degrees of activity. In addition to elastin, serine elastases have been shown to degrade or process other proteins with varying relative activities. The serine elastases share the property of preferential cleavage of polypeptides and proteins adjacent to aliphatic amino acid residues, primarily alanine and valine. These enzymes also cleave, to a variable extent, at sites adjacent to leucine and isoleucine.

Examples of serine elastases include pancreatic elastase (PE), neutrophil elastase (NE), and proteinase-3 (PR-3 or PR3).

Among elastases, the most well studied enzymes are PR-3 and NE. These enzymes are structurally similar but biologically different. Both PR-3 and NE are co-localized in neutrophil and monocyte/macrophage primary granules and are co-released from these cells when activated. Both enzymes degrade elastin when purified enzyme and substrate are incubated together. However, despite structural similarities, not all endogenous inhibitors of NE inhibit PR-3, such as, for example, secretory leukocyte protease inhibitor (SLPI) which is a potent inhibitor of NE, but has no activity against PR-3. Furthermore, the biology of NE and PR-3 appears to be significantly different.

NE appears to be primarily responsible for degradation of extracellular matrix (ECM) proteins and other important substrate proteins (immunoglobulins, surfactant apoproteins, etc.). In contrast, PR-3 appears to be particularly well-suited to the processing of pro-cytokines to their active biological forms. The amount of at least two of the more important pro-inflammatory cytokines produced by monocytic cells, TNF-α and IL-1β, has been shown to be differentially enhanced by PR-3 relative to NE. It has also been shown that PR-3, but not NE, can process mature interleukin-8 (77 amino acids) to a more potent form, interleukin-8 (70 amino acids) which has approximately 10-fold greater biological activity. Both NE and PR-3 play roles in the activation of pro-enzymes such as metalloproteinases (MMPs).

Inflammatory cell serine elastases (and metalloproteinases) are critical enzymes for directed cell migration of both neutrophils and monocyte/macrophages. Their roles in this context were thought to be limited to the degradation of vascular basement membrane and underlying extracellular matrix proteins. However, their ability to affect local regulation and amplification of the inflammatory response suggests a broader role in a variety of different disease states.

A major health challenge of the 21st Century is the rapid spread of acute and potentially highly morbid diseases such as severe acute respiratory syndrome (SARS) and avian (H5N1) influenza. The morbidity and mortality associated with these diseases is caused by the host response to the infection resulting in the well-characterized, but poorly treated, syndrome known as acute respiratory distress syndrome or ARDS. Many serious bacterial and viral infectious diseases, including avian influenza A (H5N1), have as one of their complications, the development of ALI (acute lung injury) and ARDS. Mortality in patients diagnosed with ARDS (all causes) remains approximately 35% and survivors of ARDS have persistent functional disability. In the context of H5N1 infections, this condition has a reported mortality rate of up to eighty nine percent.

Pathological findings in ARDS include diffuse alveolar and capillary injury and neutrophil-predominant inflammatory exudates in the alveolar space. Neutrophil activation leads to the release of multiple inflammatory mediators such as reactive oxygen species and proteolytic enzymes that when released in an unregulated manner cause vicinal cell injury leading to organ injury and dysfunction. One of the most important proteolytic enzymes that mediate this process is NE, which causes the general degradation of extracellular matrix proteins and other proteins such as surfactant apoproteins and non-cognate antiproteinases as well as the activation of metalloproteinase zymogens. The release of PR3 from neutrophils and monocytes enhances the activity of multiple pro-inflammatory cytokines such as TNF-α, IL-1β and IL-8 thereby amplifying the inflammatory process.

Thus, a need exists for HNE/PR3 selective inhibitors.

### SUMMARY OF THE INVENTION

The present invention provides compounds of formula (I):

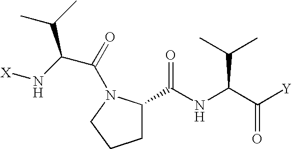

where X is R₁—(CR₃R₄)ₙOC(O)—; R₁—(CR₃R₄)ₙC(O)—; R₁—C(O)NH(CR₃R₄)ₙOC(O)—; R₁—C(O)NH(CR₃R₄)ₙC(O)—; R₁—C(O)(CR₃R₄)ₙOC(O)—; or R₁—C(O)(CR₃R₄)ₙC(O)—;

where R₁ is optionally substituted C₅₋₁₀ aryl or heteroaryl; OH or NH₂; where R₃ and R₄ are independently H or C₁₋₄ alkyl such as methyl; and

n is 0 to 6; and

Y is —CF₃ or one of:

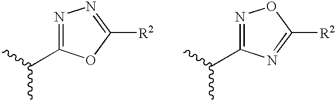

where R₂ is C₁₋₈ alkyl optionally substituted with halo or —OH; or —(CR₆R₇)ₚC₅₋₆ aryl optionally substituted with halo, —OH, C₁₋₈ alkyl, C₁₋₈ haloalkyl, —(CH₂)ₘC(O)NH₂ or —(CH₂)ₘOCH₃; where R₆ and R₇ are independently H or C₁₋₄ alkyl such as methyl; m is 0 to 4, and p is 0 or 1; or a pharmaceutically acceptable salt, ester, metabolite, or prodrug thereof.

According to several preferred embodiments, n is 0, 1 or 5. In another embodiment, R₁ is optionally substituted pyridine, phenyl or an azole such as oxazole or isoxazole. Where R₂ is alkyl, according to one embodiment it is t-butyl. In another, R₂ is phenyl optionally substituted with F or —CF₃.

In one embodiment, R₁ is

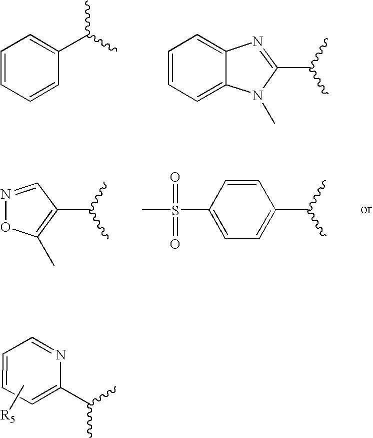

where R₅ is H, halo, OH, or NR₃R_{4,} where R₃ and R₄ are defined above.

In one preferred embodiment, X is:

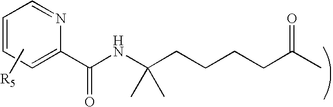

where R₅ is H, halo, or OH.

In another embodiment, R₂ is

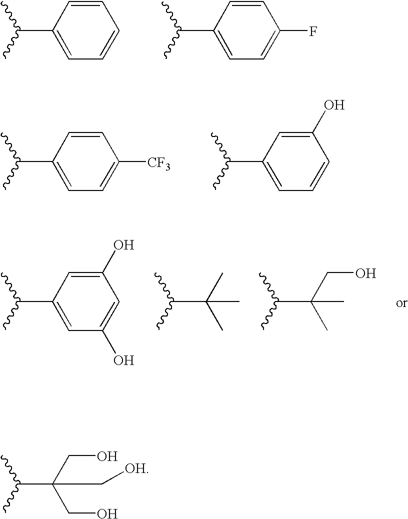

In a further embodiment, R₂ is —C(CH₃)₂—C₅₋₆ aryl, preferably phenyl, substituted with —(CH₂)ₘC(O)NH₂ or —(CH₂)ₘOCH₃—.

Preferred compounds include the following:

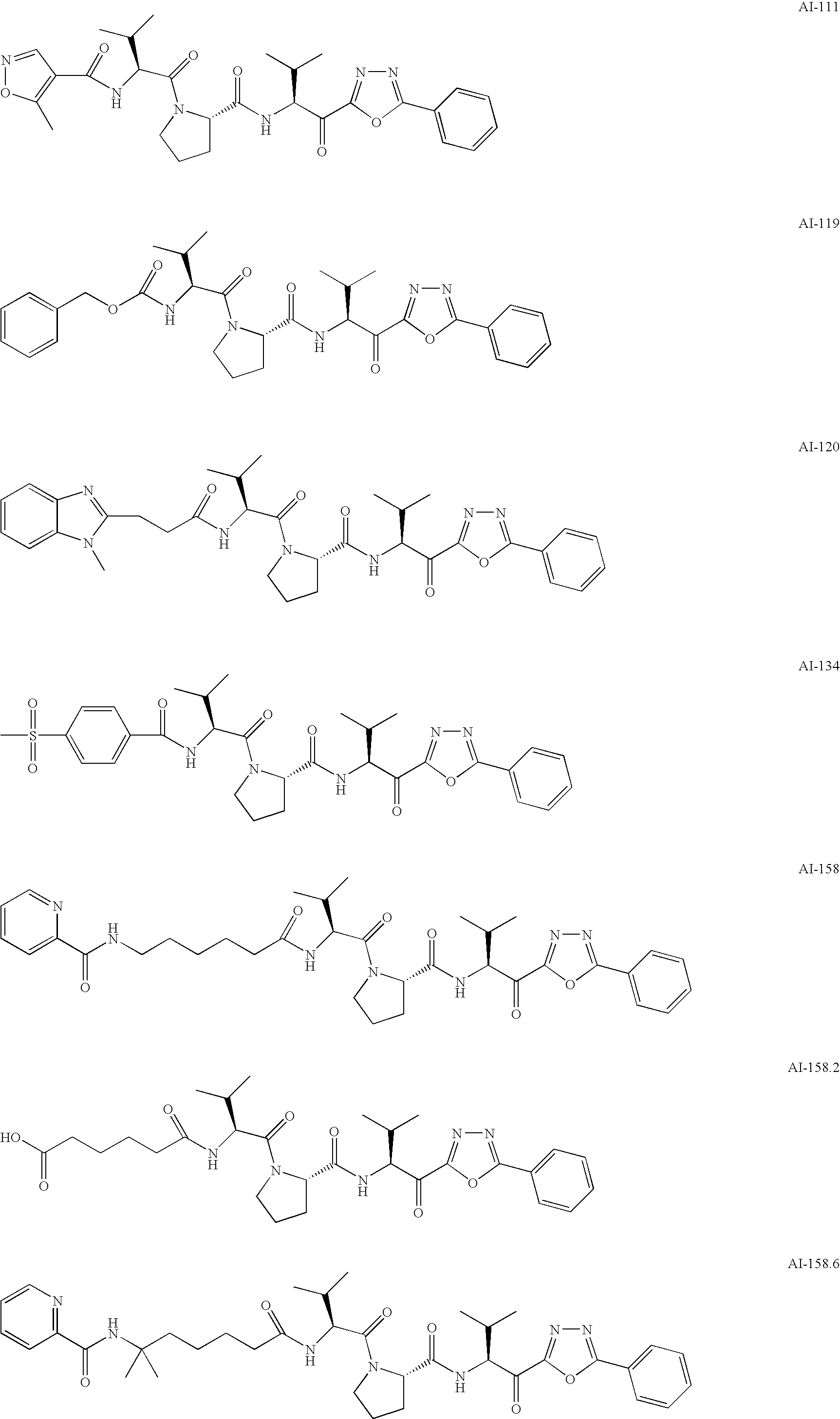

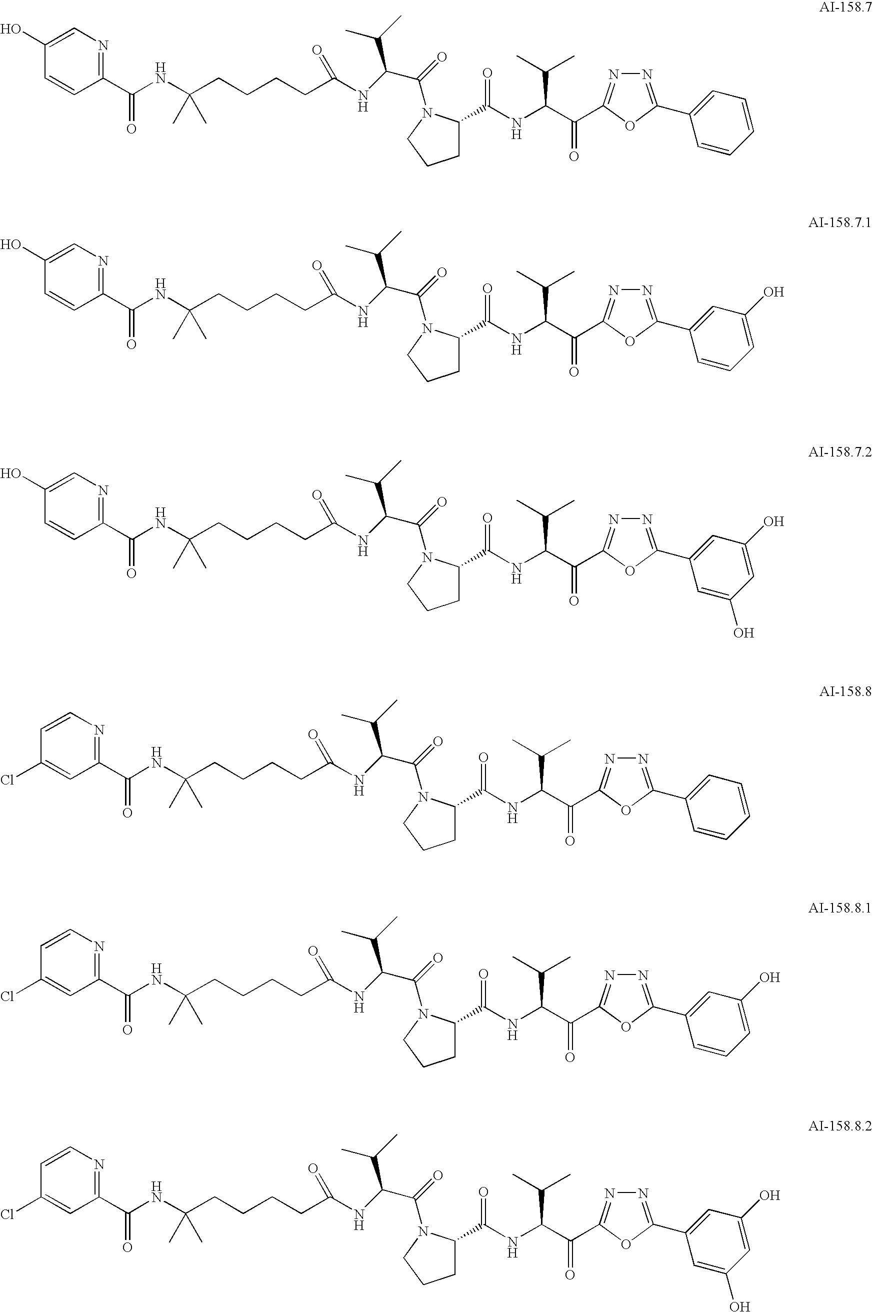

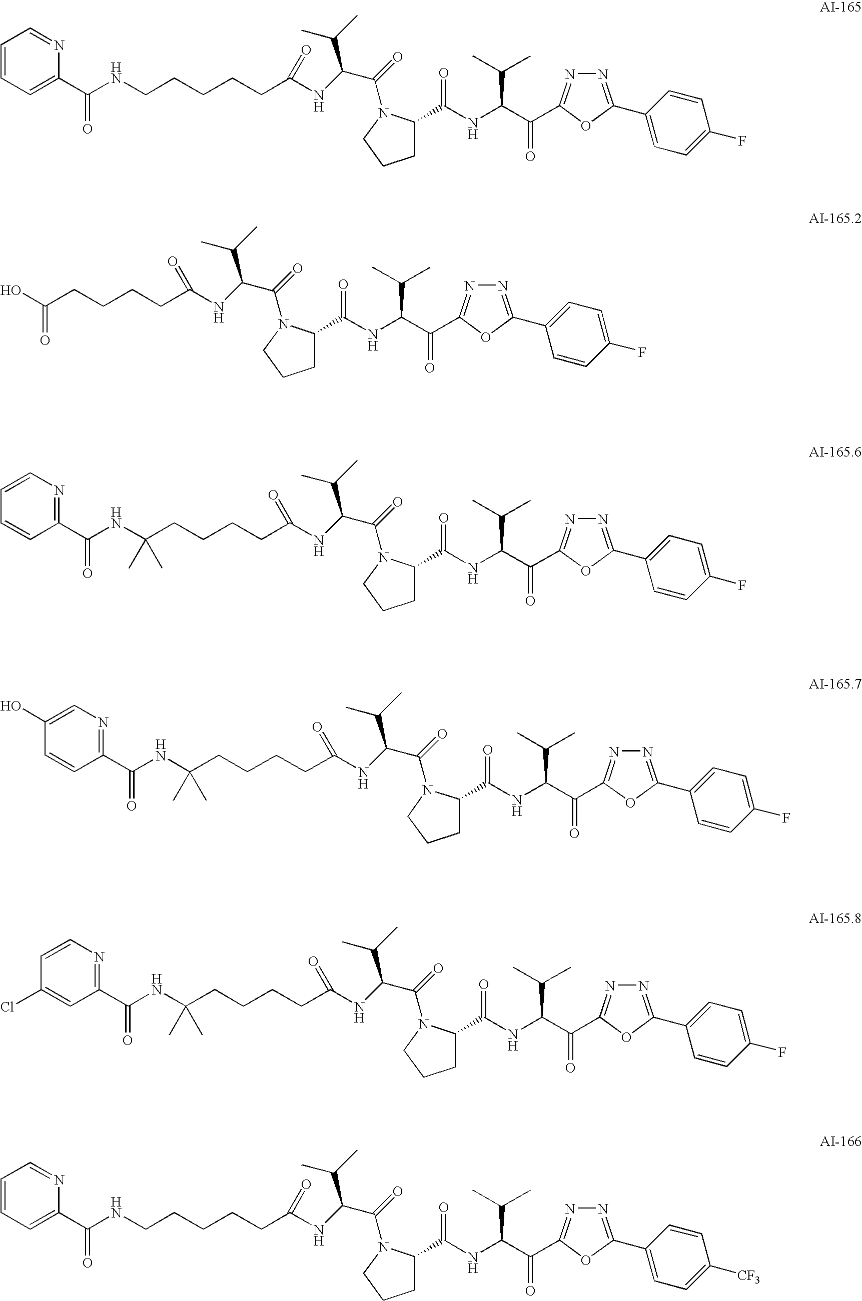

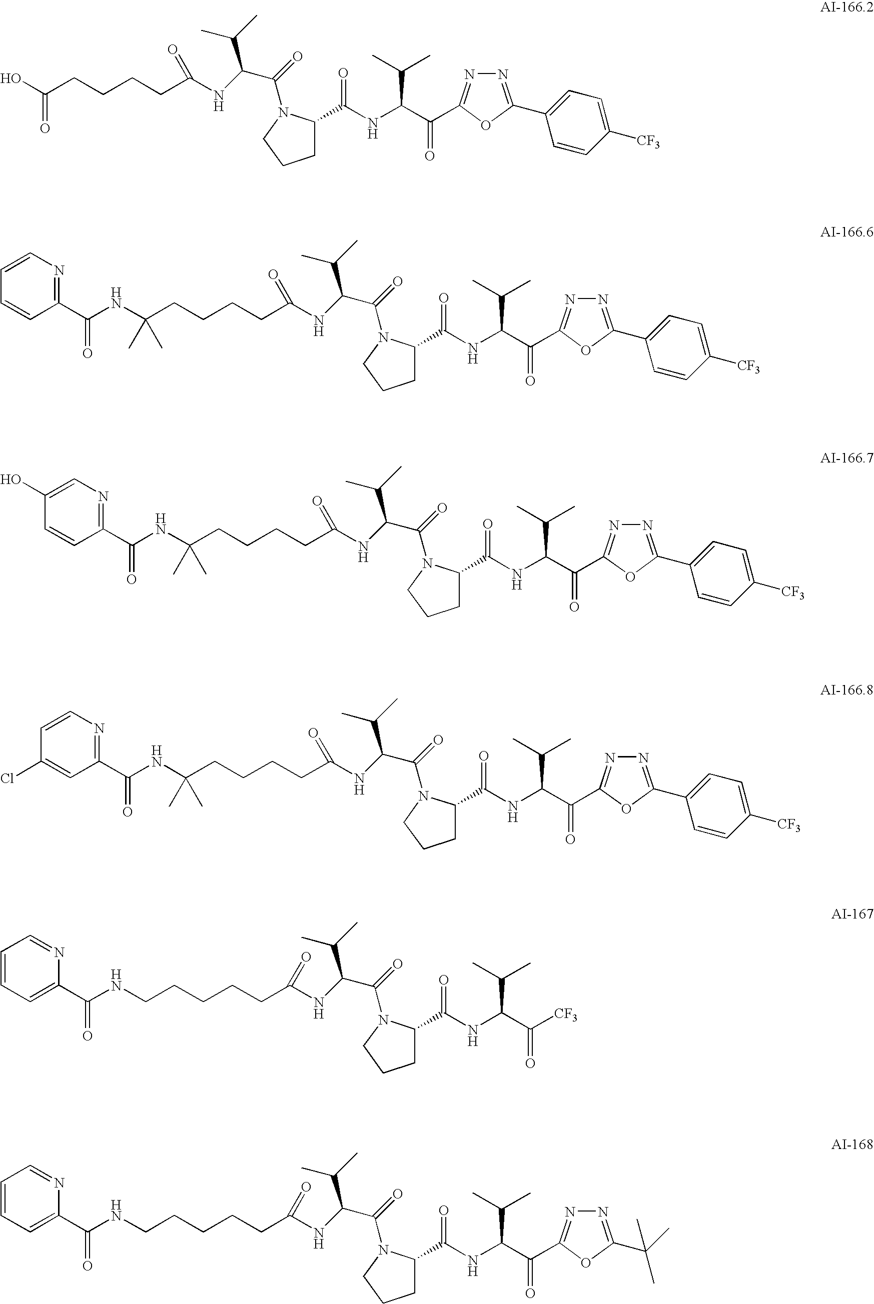

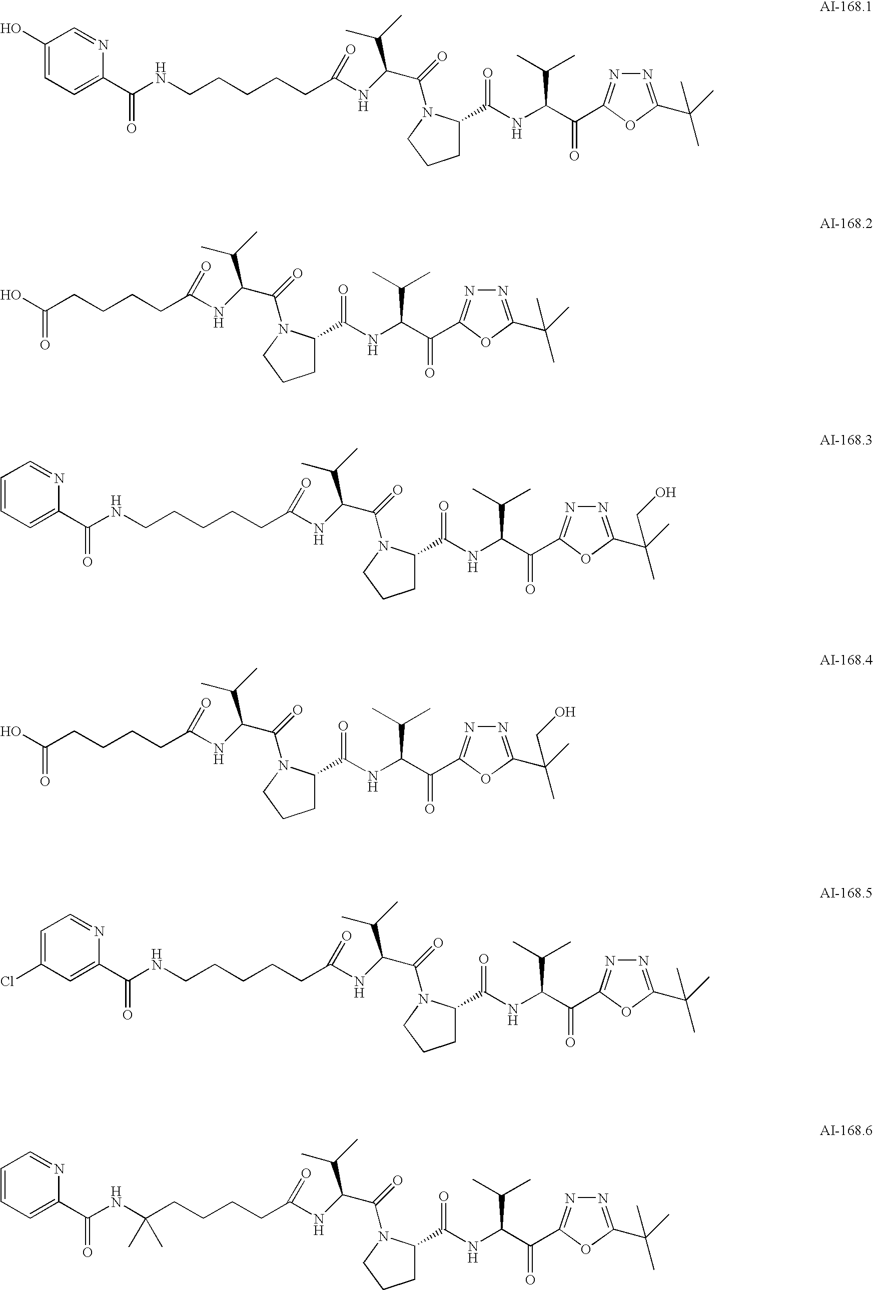

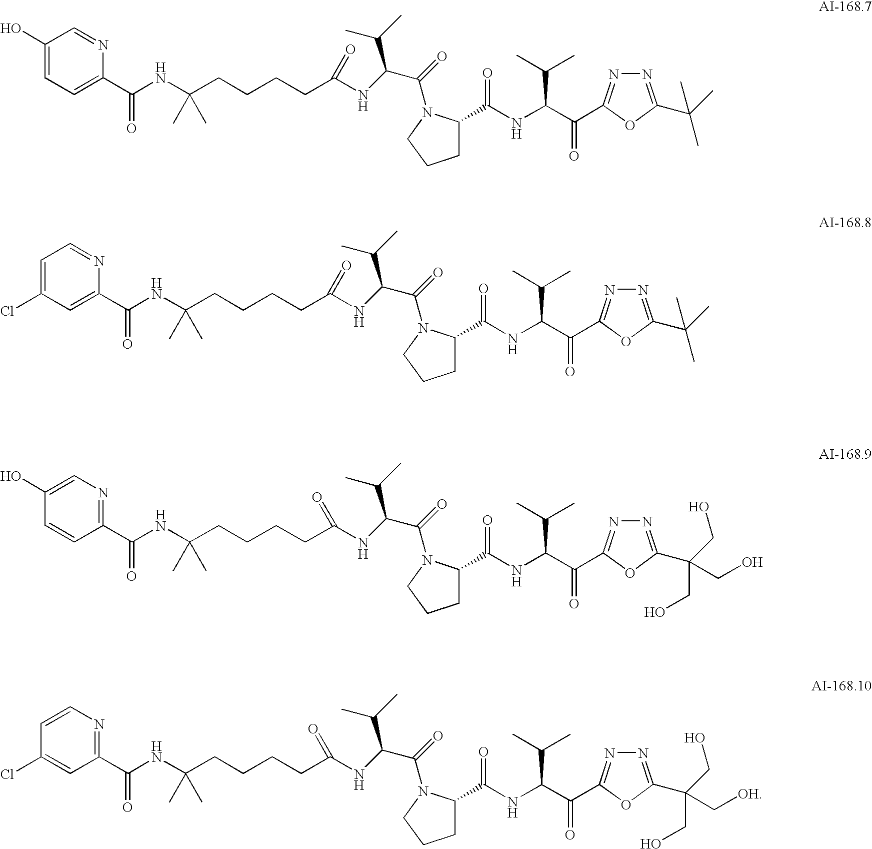

Particularly preferred compounds include:

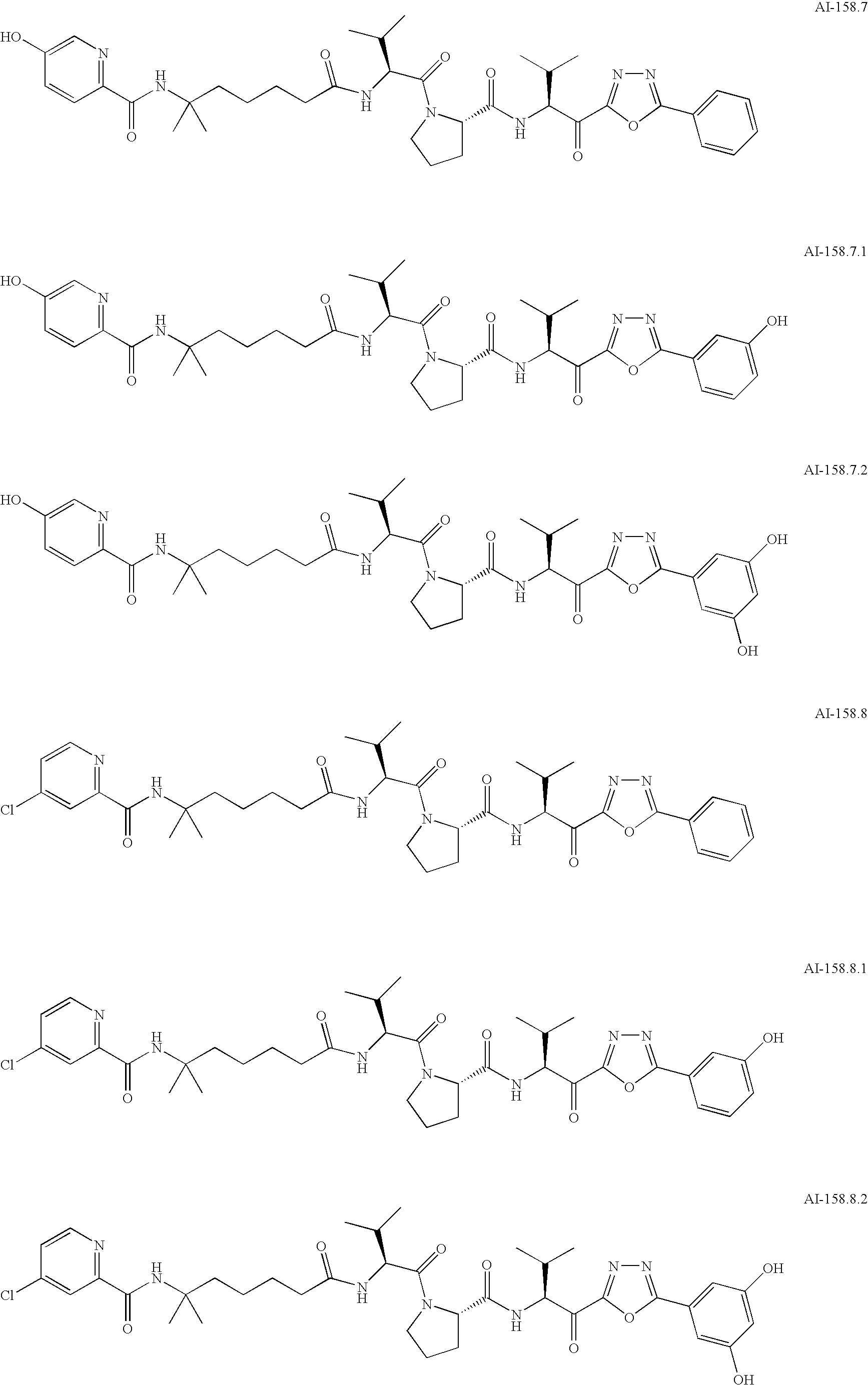

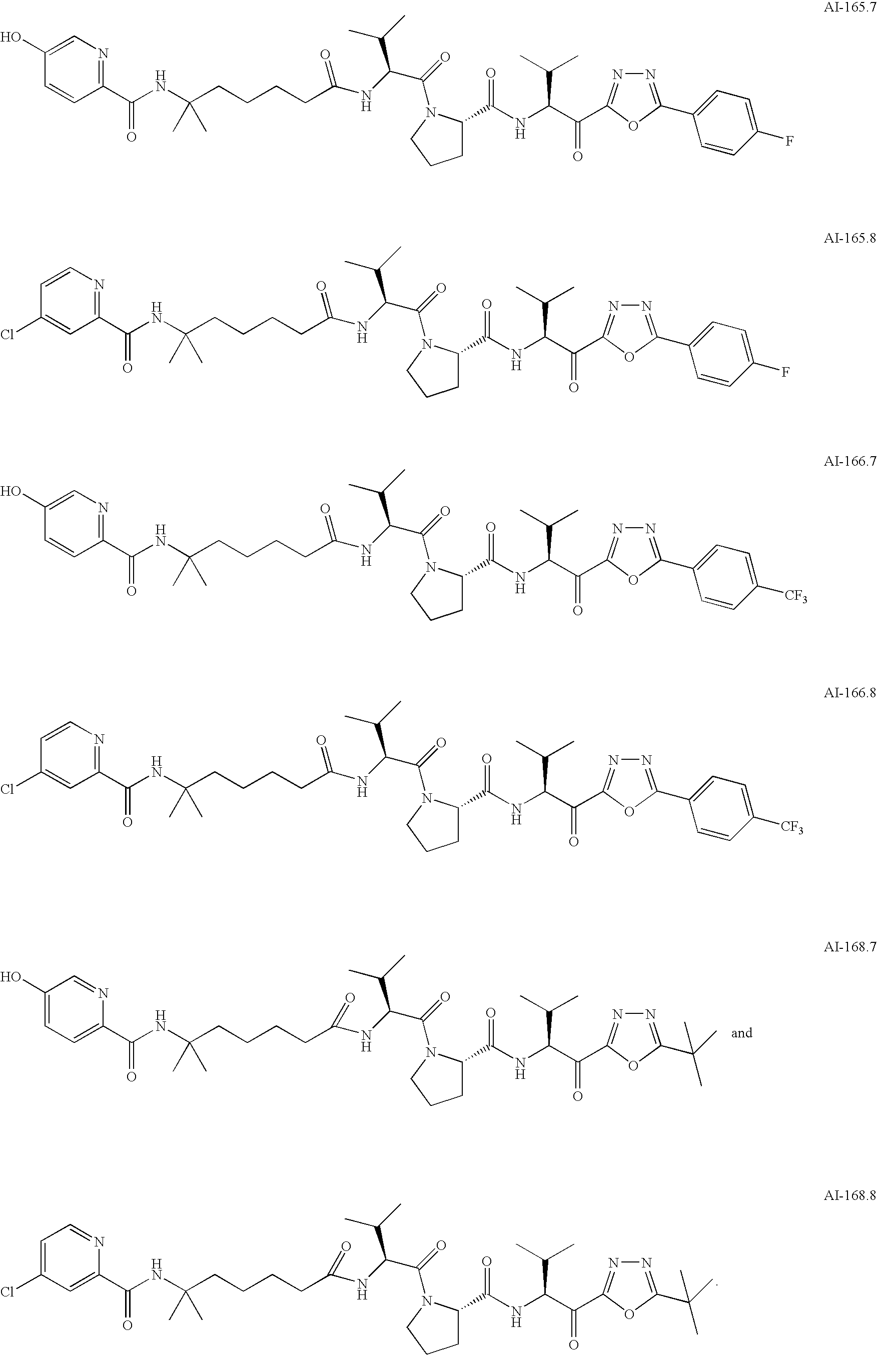

Additionally provided are pharmaceutical compositions for the inhibition of HNE and PR3 which comprise a therapeutically effective amount one or more compounds of formula (I) and a pharmaceutically acceptable carrier.

The present invention also provides methods of treatment for the inhibition of HNE and PR3 which comprises the administration to a subject in need of such inhibition a therapeutically effective amount of one or more compounds of formula (I).

### BRIEF DESCRIPTION OF THE DRAWINGS

FIG. 1 shows the binding regions of a target proteinase (top), a peptide sequence within a natural substrate (center) and a substrate based inhibitor.

FIG. 2 is a schematic showing synthetic routes used to make certain compounds of the present invention.

FIG. 3 is a schematic showing a synthetic route for preparation of compound AI-168.2.

FIG. 4 is a schematic showing a synthetic route for preparation of compound AI-168.7.

FIG. 5 is a schematic showing a synthetic route for preparation of compound AI-168.8.

FIG. 6 shows a proposed metabolic pathway for compound AI-168.

### DETAILED DESCRIPTION OF THE INVENTION

As used herein, the following definitions shall apply unless otherwise indicated.

The phrase “optionally substituted” is used interchangeably with the phrase “substituted or unsubstituted.” Unless otherwise indicated, an optionally substituted group may have a substituent at each substitutable position of the group, and each substitution is independent of any other. Also, combinations of substituents or variables are permissible only if such combinations result in stable compounds. In addition, unless otherwise indicated, functional group radicals are independently selected. Where “optionally substituted” modifies a series of groups separated by commas (e.g., “optionally substituted A, B or C”; or “A, B or C optionally substituted with”), it is intended that each of the groups (e.g., A, B and C) is optionally substituted.

The phrase “HNE/PR3 selective” or “selectivity” means that the ratio of Ki's for HNE and PR3 for a particular compound are within a factor of about 1,000, and more preferably within about 100, of each other and, preferably, that the Ki's are about 10,000 fold less potent for other serine proteinases such as trypsin, chymotrypsin, and Cathepsin G. Determination of Ki may be achieved using means known in the art, such as the methods disclosed in the Examples (see also, Wieczorek et al., Archives Biochem. and Biophysics, 1999, 367:193-201).

The term “HNE” means human neutrophil elastase (see Bode et al., Biochemistry, 1989, 28: 1951-1963; Bernstein et al., Med. Res. Rev., 1994, 14: 127-194)

The term “PR3” means human neutrophil proteinase 3/myoblastin (see Rao et al., J. Biol. Chem. 1991, 266(15): 9540-9548.)

The term “aliphatic” or “aliphatic group” as used herein means a straight-chain or branched C1-12 hydrocarbon chain that is completely saturated or that contains one or more units of unsaturation, or a monocyclic C3-8 hydrocarbon or bicyclic C8-12 hydrocarbon that is completely saturated or that contains one or more units of unsaturation, but which is not aromatic (also referred to herein as “carbocycle” or “cycloalkyl”), that has a single point of attachment to the rest of the molecule wherein any individual ring in said bicyclic ring system has 3-7 members. For example, suitable aliphatic groups include, but are not limited to, linear or branched alkyl, alkenyl, alkynyl groups and hybrids thereof such as (cycloalkyl)alkyl, (cycloalkenyl)alkyl or (cycloalkyl)alkenyl.

The terms “alkyl,” “alkoxy,” “hydroxyalkyl,” “alkoxyalkyl” and “alkoxycarbonyl,” used alone or as part of a larger moiety include both straight and branched chains containing one to twelve carbon atoms. The terms “alkenyl” and “alkynyl” used alone or as part of a larger moiety shall include both straight and branched chains containing two to twelve carbon atoms.

The terms “haloalkyl,” “haloalkenyl” and “haloalkoxy” means alkyl, alkenyl or alkoxy, as the case may be, substituted with one or more halogen atoms. The term “halogen” or “halo” means F, Cl, Br or I.

The term “heteroatom” means nitrogen, oxygen, or sulfur and includes any oxidized form of nitrogen and sulfur, and the quaternized form of any basic nitrogen.

The term “aryl” used alone or in combination with other terms, refers to monocyclic, bicyclic or tricyclic carbocyclic ring systems having a total of five to fourteen ring members, wherein at least one ring in the system is aromatic and wherein each ring in the system contains 3 to 8 ring members. The term “aryl” may be used interchangeably with the term “aryl ring”. The term “aralkyl” refers to an alkyl group substituted by an aryl. The term “aralkoxy” refers to an alkoxy group substituted by an aryl.

As used herein, where a ring is defined to contain or comprise x to y members, it is understood that the total number of member atoms (e.g., carbon or heteroatoms) making up the ring is x, y or any integer between x and y. By way of example, a ring comprising 3 to 8 carbon or heteroatoms may be a ring containing 3, 4, 5, 6, 7 or 8 ring members.

The term “heterocycloalkyl,” “heterocycle,” “heterocyclyl” or “heterocyclic” as used herein means monocyclic, bicyclic or tricyclic ring systems having 5, 6, 7, 8, 9, 10, 11, 12, 13 or 14 ring members in which one or more ring members is a heteroatom, wherein each ring in the system contains 3, 4, 5, 6, 7 or 8 ring members and is non-aromatic.

The term “heteroaryl,” used alone or in combination with other terms, refers to monocyclic, bicyclic and tricyclic ring systems having a total of 5, 6, 7, 8, 9, 10, 11, 12, 13 or 14 ring members, and wherein: 1) at least one ring in the system is aromatic; 2) at least one ring in the system contains one or more heteroatoms; and 3) each ring in the system contains 3, 4, 5, 6 or 7 ring members. The term “heteroaryl” may be used interchangeably with the term “heteroaryl ring” or the term “heteroaromatic”. Examples of heteroaryl rings include, but are not limited to, 2-furanyl, 3-furanyl, N-imidazolyl, 2-imidazolyl, 4-imidazolyl, 5-imidazolyl, 3-isoxazolyl, 4-isoxazolyl, 5-isoxazolyl, 2-oxadiazolyl, 5-oxadiazolyl, 2-oxazolyl, 4-oxazolyl, 5-oxazolyl, 1-pyrrolyl, 2-pyrrolyl, 3-pyrrolyl, 1-pyrazolyl, 3-pyrazolyl, 4-pyrazolyl, 2-pyridyl, 3-pyridyl, 4-pyridyl, 2-pyrimidyl, 4-pyrimidyl, 5-pyrimidyl, 3-pyridazinyl, 2-thiazolyl, 4-thiazolyl, 5-thiazolyl, 5-tetrazolyl, 2-triazolyl, 5-triazolyl, 2-thienyl, 3-thienyl, carbazolyl, benzimidazolyl, benzothienyl, benzofuranyl, indolyl, quinolinyl, benzotriazolyl, benzothiazolyl, benzooxazolyl, benzimidazolyl, isoquinolinyl, indazolyl, isoindolyl, acridinyl, and benzoisoxazolyl. The term “heteroaralkyl” refers to an alkyl group substituted by a heteroaryl. The term “heteroarylalkoxy” refers to an alkoxy group substituted by a heteroaryl.

An aryl (including aralkyl, aralkoxy, aryloxyalkyl and the like) or heteroaryl (including heteroaralkyl, heteroarylalkoxy and the like) group may contain one or more substituents. Suitable substituents on an unsaturated carbon atom of an aryl, heteroaryl, aralkyl or heteroaralkylgroup are selected from halogen; haloalkyl; —CF₃; —R; —OR; —SR; 1,2-methylenedioxy; 1,2-ethylenedioxy; protected OH (such as acyloxy); phenyl (Ph); Ph substituted with R; —O(Ph); —O—(Ph) substituted with R; —CH₂(Ph); —CH₂(Ph) substituted with R; —CH₂CH₂(Ph); —CH₂CH₂(Ph) substituted with R; —NO₂; —CN; —N(R)₂; —NRC(O)R; —NRC(O)N(R)₂; —NRCO₂R; —NRNRC(O)R; —NR—NRC(O)N(R)₂; —NRNRCO₂R; —C(O)C(O)R; —C(O)CH₂C(O)R; —CO₂R; —C(O)R; —C(O)N(R)₂; —OC(O)N(R)₂; —S(O)₂R; —SO₂N(R)₂; —S(O)R; —NRSO₂N(R)₂; —NRSO₂R; —C(=S)N(R)₂; —C(═NH)—N(R)₂; —(CH₂)_{y} NHC(O)R; —(CH₂)_{y}R; —(CH₂)_{y}NHC(O)NHR; —(CH₂)_{y}NHC(O)OR; —(CH₂)_{y}NHS(O)R; —(CH₂)_{y}NHSO₂R; —(CH₂)_{y}NHC(O)CH((V)_{z}—R)(R); —(CH₂)ₘC(O)NH₂ or —(CH₂)ₘOCH₃ wherein each R is independently selected from hydrogen, optionally substituted aliphatic (preferably C₁₋₆), an unsubstituted heteroaryl or heterocyclic ring (preferably C₅₋₆), phenyl (Ph), —O(Ph), or —CH₂(Ph)-CH₂(Ph), wherein m is 0-4; y is 0-6; z is 0-1; and V is a linker group. When R is aliphatic, it may be substituted with one or more substituents selected from —NH₂, —NH(C₁₋₄ aliphatic), —N(C₁₋₄ aliphatic)₂, —S(O)(C₁₋₄ aliphatic), —SO₂(C₁₋₄ aliphatic), halogen (C₁₋₄ aliphatic), —OH, —O—(C₁₋₄ aliphatic), —NO₂, —CN, —CO₂H, —CO₂(C₁₋₄ aliphatic), —O(halo C₁₋₄ aliphatic) or -halo(C₁₋₄ aliphatic); wherein each C₁₋₄ aliphatic is unsubstituted.

An aliphatic group or a non-aromatic heterocyclic ring may contain one or more substituents. Suitable substituents on a saturated carbon of an aliphatic group or of a non-aromatic heterocyclic ring are selected from those listed above for the unsaturated carbon of an aryl or heteroaryl group and the following: ═O, ═S, ═NNHR, ═NN(R)₂, ═N—, ═NNHC(O)R, ═NNHCO₂(alkyl), ═NNHSO₂(alkyl), or ═NR, where each R is independently selected from hydrogen or an optionally substituted aliphatic (preferably C₁₋₆). When R is aliphatic, it may be substituted with one or more substituents selected from —NH₂, —NH(C₁₋₄ aliphatic), —N(C₁₋₄ aliphatic)₂, halogen, —OH, —O—(C₁₋₄ aliphatic), —NO₂, —CN, —CO₂H, —CO₂(C₁₋₄ aliphatic), —O(halo C₁₋₄ aliphatic), or -halo(C₁₋₄ aliphatic); wherein each C₁₋₄ aliphatic is unsubstituted.

Substituents on a nitrogen of a non-aromatic heterocyclic ring are selected from —R, —N(R)₂, —C(O)R, —C(O)OR, —C(O)C(O)R, —C(O)CH₂C(O)R, —SO₂R, —SO₂N(R)₂, —C(═S)N(R)₂, —C(═NH)—N(R)₂ or —NRSO₂R; wherein each R is independently selected from hydrogen, an optionally substituted aliphatic (preferably C₁₋₆), optionally substituted phenyl (Ph), optionally substituted -O(Ph), optionally substituted —CH₂(Ph), optionally substituted —CH₂CH₂(Ph), or an unsubstituted heteroaryl or heterocyclic ring (preferably 5-6 membered). When R is a C₁₋₆ aliphatic group or a phenyl ring, it may be substituted with one or more substituents selected from —NH₂, —NH(C₁₋₄ aliphatic), —N(C₁₋₄ aliphatic)₂, halogen, —(C₁₋₄ aliphatic), —OH, —O—(C₁₋₄ aliphatic), —NO₂, —CN, —CO₂H, —CO₂(C₁₋₄ aliphatic), —O(halo C₁₋₄ aliphatic) or -halo(C₁₋₄ aliphatic); wherein each C₁₋₄ aliphatic is unsubstituted.

The term “treatment” refers to any treatment of a pathologic condition in a mammal, particularly a human, and includes: (i) preventing the pathologic condition from occurring in a subject which may be predisposed to the condition but has not yet been diagnosed with the condition and, accordingly, the treatment constitutes prophylactic treatment for the disease condition; (ii) inhibiting the pathologic condition, i.e., arresting its development; (iii) relieving the pathologic condition, i.e., causing regression of the pathologic condition; or (iv) relieving the conditions mediated by the pathologic condition.

The term “therapeutically effective amount” refers to that amount of a compound of the invention that is sufficient to effect treatment, as defined above, when administered to a mammal in need of such treatment. The therapeutically effective amount will vary depending upon the subject and disease condition being treated, the weight and age of the subject, the severity of the disease condition, the manner of administration and the like, which can readily be determined by one of ordinary skill in the art.

The term “pharmaceutically acceptable salts” includes, but is not limited to, salts well known to those skilled in the art, for example, mono-salts (e.g. alkali metal and ammonium salts) and poly salts (e.g. di- or tri-salts,) of the compounds of the invention. Pharmaceutically acceptable salts of compounds of Formula (I) are where, for example, an exchangeable group, such as hydrogen in —OH, —NH—, or —P(═O)(OH)—, is replaced with a pharmaceutically acceptable cation (e.g. a sodium, potassium, or ammonium ion) and can be conveniently be prepared from a corresponding compound of Formula (I) by, for example, reaction with a suitable base. In cases where compounds are sufficiently basic or acidic to form stable nontoxic acid or base salts, administration of the compounds as salts may be appropriate. Examples of pharmaceutically acceptable salts are organic acid addition salts formed with acids that form a physiological acceptable anion, for example, tosylate, methanesulfonate, acetate, citrate, malonate, tartarate, succinate, benzoate, ascorbate, α-ketoglutarate, and α-glycerophosphate. Suitable inorganic salts may also be formed, including hydrochloride, sulfate, nitrate, bicarbonate, and carbonate salts. Pharmaceutically acceptable salts may be obtained using standard procedures well known in the art, for example, by reacting a sufficiently basic compound such as an amine with a suitable acid affording a physiologically acceptable anion. Alkali metal (for example, sodium, potassium or lithium) or alkaline earth metal (for example, calcium) salts of carboxylic acids can also be made.

The term “prodrug” or “prodrugs” is used in its ordinary meaning in the art and means a compound of the invention that has its charged moieties masked or protected by another moiety that is designed to be cleaved under particular physiological conditions, leaving the deprotected or unmasked compound of the invention. The use of masking agents is common and well-known in the art and, in particular, masking phosphate or phosphonate groups. All such masking agents are suitable and can be used with the compounds of the invention. Various agents such as acyloxy alkyl esters are described by Srivasta et al., (1984 Bioorganic Chemistry 12, 118-12), and by Freeman et al. (1997 Progress in Medicinal Chemistry 34:112-147) which are each incorporated in their entirety herein by reference; and 3-phthalidyl phosphonate esters are described by Dang Q., et al., (1999 Bioorganic & Med. Chem Letters, 9:1505-1510), which is incorporated in its entirety herein by reference. For example, and not by way of limitation, Srivasta et al. also describe acetoxymethyl, isobutryloxymethyl, and pivaloxymethyl as masking agents. Other suitable masking groups comprising pivaloxyalkyl, e.g., pivaloxymethyl, or a pivaloyloxy group as described by Farquhar D. et al., (1995 J. Med. Chem., 38:488-495) which is incorporated in its entirety herein by reference. Still other masking or protecting agents are described in U.S. Pat. Nos. 4,816,570 and 4,968,788 both of which are incorporated in their entirety herein by reference. Lipid prodrugs are also suitable for use with the compounds of the invention. By non-limiting example, certain lipid prodrugs are described in Hostetler et al., (1997 Biochem. Pharm. 53:1815-1822), and Hostetler et al., 1996 Antiviral Research 31:59-67), both of which are incorporated in their entirety herein by reference. Additional examples of suitable prodrug technology is described in WO 90/00555; WO 96/39831; WO 03/095665A2; U.S. Pat. Nos. 5,411,947; 5,463,092; 6,312,662; 6,716,825; and U.S. Published Patent Application Nos. 2003/0229225 and 2003/0225277 each of which is incorporated in their entirety herein by reference. Such prodrugs may also possess the ability to target the drug compound to a particular tissue within the patient, e.g., liver, as described by Erion et al., (2004 J. Am. Chem. Soc. 126:5154-5163; Erion et al., Am. Soc. Pharm. & Exper. Ther. DOI: 10.1124/jept.104.75903 (2004); WO 01/18013 A1; U.S. Pat. No. 6,752,981), each of which is incorporated in their entirety herein by reference. By way of non-limiting example, other prodrugs suitable for use with the compounds of the invention are described in WO 03/090690; U.S. Pat. No. 6,903,081; U.S. Patent Application No. 2005/0171060A1; U.S. Patent Application No. 2002/0004594A1; and by Harris et al., (2002 Antiviral Chem. & Chemo. 12: 293-300; Knaggs et al., 2000 Bioorganic & Med. Chem. Letters 10: 2075-2078) each of which is incorporated in their entirety herein by reference.

Some of the compounds described herein possess one or more chiral (also known as asymmetric) centers, and may lead to optical isomers. All such isomers, as well as diastereomers and enantiomers are included in the present invention. Racemic mixtures of compounds are also included in the present invention. Resolution of such racemic mixtures can be made using standard procedures known in the art. By way of non-limiting example, one of skill in the art can obtain the two enantiomers of the racemic amino acid by using chiral column separation or by proper functionalization followed by enzymatic resolution or by treatment of the racemate with a chiral amine to form a diastereomeric salt and the two diastereomers separated by crystallization. The parent compound can then be liberated from the amine salt by acid treatment. Alternatively, one can obtain the two enantiomers of the racemic final compound by using chiral column separation or by treatment with a chiral amine to form a diastereomeric salt and the two diastereomers separated by crystallization. The parent compound can then be liberated from the amine salt by acid treatment. Another method that can be used to resolve enantiomers of a chiral amino acid is to form a conjugate (e.g. ester) with a chiral moiety (e.g. a chiral alcohol) to produce a mixture of diasteromeric adducts. These adducts can be separated by ordinary (non-chiral) chromatography or by fractional crystallization, then the respective enantiomers of the amino acid liberated by cleavage of the conjugate.

HNE and PR3 are co-localized and co-released from the azurophilic granules of the neutrophil and certain monocytes subsets. They are also inhibited by the same host anti-proteinases (alpha-1 proteinase inhibitor and elafin). The present invention is based on the proposition that inhibiting these two proteinases simultaneously with a single chemical entity would provide a significant clinical benefit, if the compound had relatively balanced inhibitory activity with respect to these two targets and was without significant or selective activity against other serine proteinases.

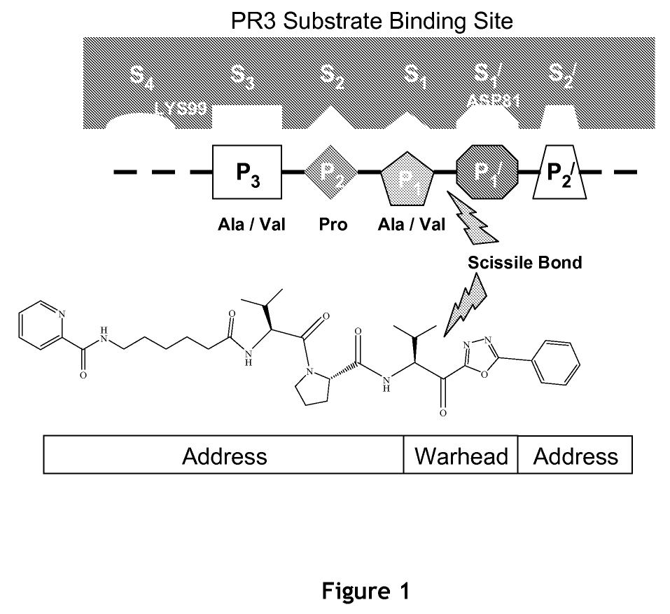

Illustrated in FIG. 1 is the accepted understanding and nomenclature of how proteinases bind to their target substrates (and substrate based inhibitors). The compounds disclosed herein take advantage of known substrate binding motifs that are specific to serine elastases (HNE and PR3). However, these motifs are modified in the P4 and P1′ regions in order to enhance activity of the inhibitors with respect to PR3 while still retaining significant potency against HNE.

FIG. 1 shows the binding regions of a target proteinase (top), a peptide sequence within a natural substrate (center) and a substrate based inhibitor designed to take advantage of the known enzyme—substrate binding interactions. The peptide bond that is cleaved by the proteinase (the scissile bond) is identified by the “lightning” symbol. The amino acid immediately to the left (towards the amino terminus of the protein) is identified as P1. The second amino acid from the scissile bond is designated P2, and so forth. The amino acid immediately to the right (towards the carboxy terminus of the protein) is designated P1′, the second is designated P2′, etc. The corresponding subsites within the binding region of the protein are designated S1, S2, S3, etc. and S1′, S2′, S3′, etc. In general, the specificity of serine proteinases is determined by S-P interactions, with S1-P1 binding being the most important for determining the specificity of the enzyme for its targets. For example, all serine elastases require small linear aliphatic amino acids (alanine, valine, etc.) in the P1 region; all trypsin-like enzymes require basic amino acids (lysine or arginine) in the P1 region and all chymotrypsin-like enzymes require aromatic amino acids (phenylalanine or tyrosine) in the P1 region. Further specificity within each class of serine proteinases is provided by other S-P or S′-P′ interactions. Substrate-based inhibitors take advantage of these enzyme-substrate interactions by using known substrate binding motifs (addresses) to target specific enzymes and reduce their interactions with other non-target proteinase by several orders of magnitude. One strategy for inhibition involves use of specific “addresses” to bring a warhead (a specific chemical moiety known to interfere with the catalytic mechanism of the enzyme) into the binding site of the target enzyme, thereby inhibiting the catalytic activity of the proteinase. In the compounds disclosed herein, “addresses” (P1-P3) known to target serine elastases are specifically modified in the P4 and P1′ regions of the molecule to enhance activity against PR3 relative to HNE while still retaining potent HNE inhibitory activity.

In order to discern differences between PR3 and HNE, the crystal structure of PR3 was superimposed on the crystal structure of HNE. The serine-histidine-aspartic acid catalytic triad of these serine elastases was used to orient the two enzymes. In this way, subtle differences in the substrate binding regions of the two proteins were identified. Key differences between PR3 and HNE were identified at specific amino acid positions.

Ile 180 in PR3 limits the size and shape of the P1 pocket when compared to Val 180 in HNE. Immediately to the lower left of the catalytic triad is the S1 subsite of the enzyme in which the first difference between the two enzymes is noted. In HNE, the residue forming the lower left boundary of this subsite is valine. In PR3, it is isoleucine.

Lys 99 in PR3, compared to Leu 99 in HNE, defines S2/S4-P2/P4 interactions. Another difference is in the S2-S4 region of these enzymes (up and to the left of the catalytic triad). In PR3, the residue forming the upper border of these two subsites is lysine. In HNE, it is leucine. However, the side chain of this residue is free to move between these two subsites based on the structure of the substrate or inhibitor bound to the enzyme. In certain preferred embodiments, the compounds of the present invention incorporate a hydrophobic residue (alanine, valine or cyclobutyl glycine) in the P3 region of the inhibitor. This forces the lysine side chain of PR3 into the S4 binding region. The R3 modifications illustrated in FIG. 1 are designed to form hydrogen bonds with the lysine side chain, while still preserving the overall hydrophobic nature of the compound.

Asp 81 in PR3, compared to Asn 81 in HNE, delineates S1′-P1′ substrate-enzyme interactions. A further distinction, illustrated by the two residues to the upper right of the catalytic triad, involves the residues forming the upper border of the S1′. In PR3, this residue is an aspartic acid. In HNE, it is an asparagine. As with the R3 modifications, the R4 modifications shown in FIG. 1 are designed to provide hydrogen bond interactions with the aspartic acid in PR3 while still preserving the overall hydrophobic nature of the compounds, which is required for overall serine elastase inhibitory activity.

Another difference between PR3 and HNE (to the left of the P1 binding site) was identified in the crystal structure superimposition of PR3 on HNE; however, the difference is embedded within the proteins and does not interact with either substrates or inhibitors.

The structure of CE-2072, which is used in comparative studies, is set forth below.

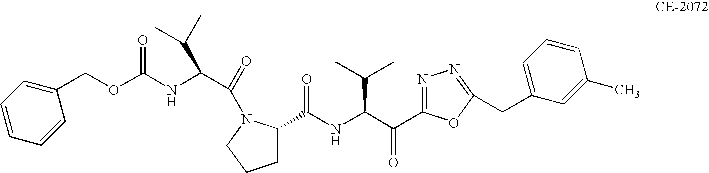

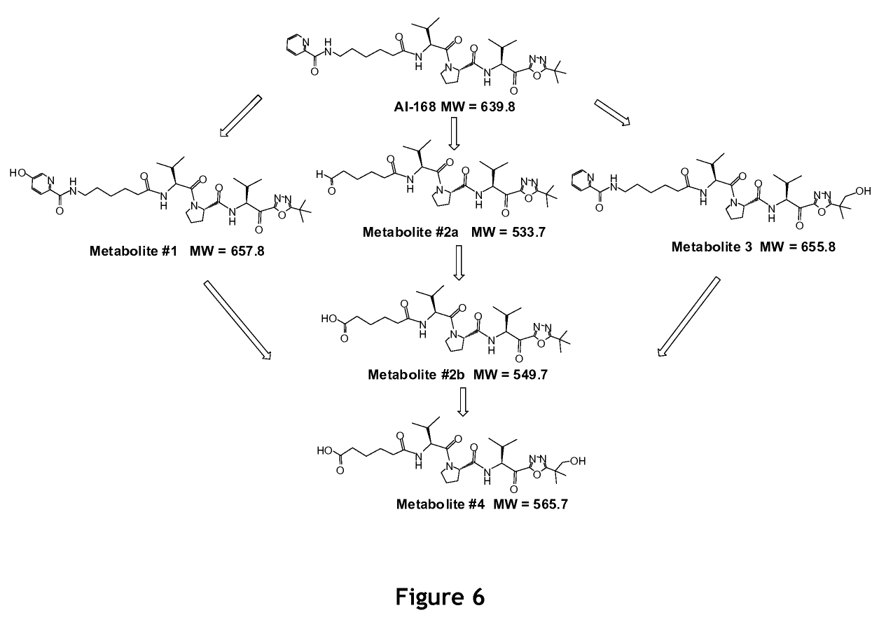

The serine elastase inhibitors of the present invention include compounds of formula (I). Certain preferred compounds of formula (I) include AI-158 and AI-168. In one aspect, compounds of the present invention include metabolites of the compounds of formula (I). For example, metabolites of compound AI-168 are identified in Example 7 and are shown in FIG. 6. In this aspect, preferred compounds include AI-168.2. In another aspect, certain preferred compounds of formula (I) are provided which improve metabolic stability and therefore enhance utility as pharmaceuticals. In this aspect, preferred compounds of formula (I) include AI-158.7, AI-158.7.1, AI-158.7.2, AI-158.8, AI-158.8.1, AI-158.8.2, AI-165.7, AI-165.8, AI-166.7, AI-166.8, AI-168.7 and AI-168.8.

In one aspect, compounds of formula (I) wherein X is

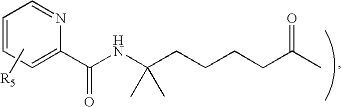

where R₅ is H, halo, or OH, are found to exhibit improved activity against PR3 as well as improved water solubility.

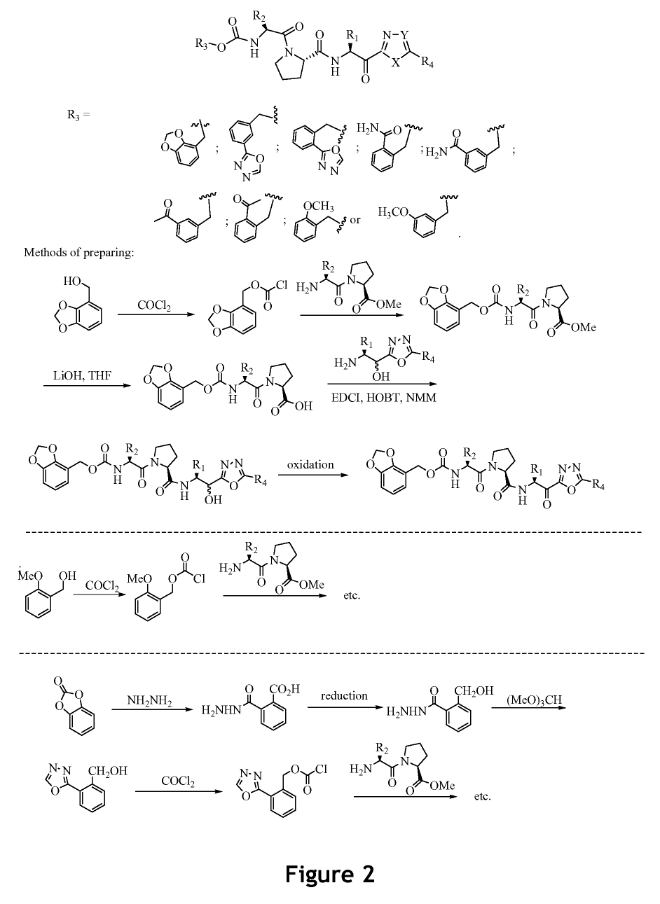

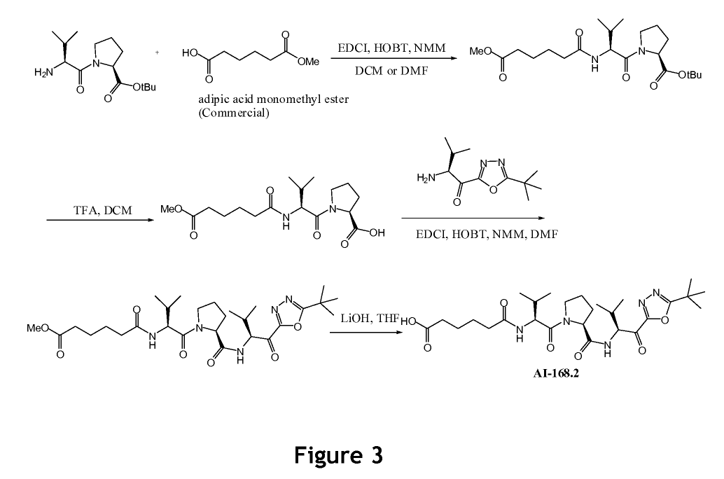

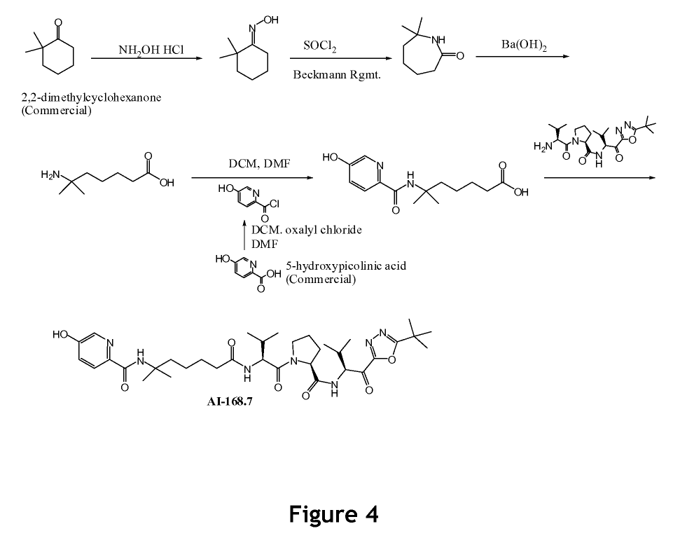

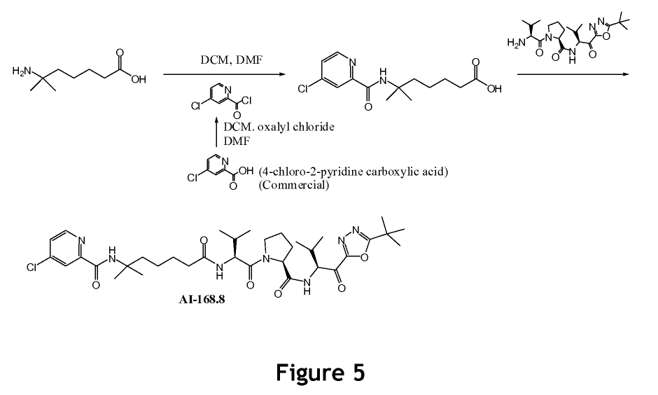

The compounds of the present invention may be prepared by any means known to those skilled in the art. Exemplary schematics are set forth in FIGS. 2-5. Additional methods are disclosed in the art, for example, in U.S. Pat. No. 5,801,148, which is incorporated herein by reference. Specific methods for certain preferred embodiments are set forth in the Examples below. Specific synthetic routes for preferred compounds AI-168.2, AI-168.7 and AI-168.8 are set forth in FIGS. 3-5.

The compounds of the present invention may be used to inhibit HNE and PR3, and preferably both. The compounds can be used to reduce inflammation and/or relieve pain in diseases such as emphysema, rheumatoid arthritis, osteoarthritis, gout, bronchial inflammation, chronic or acute bronchitis, cystic fibrosis, adult respiratory distress syndrome, atherosclerosis, sepsis, septicemia, shock, periodontitis, glomerular nephritis or nephosis, myocardial infarction, reperfusion injury, infectious arthritis, rheumatic fever and the like, and may reduce hemorrhage in acute promyelocytic leukemia and the like. The compounds may be used to treat the disease and symptoms associated with serious bacterial and viral infectious diseases, such as SARS and avian influenza, including ALI and ARDS.

Dosage levels on the order of from about 0.01 mg to about 100 mg/kg of body weight per day are useful in the treatment of the above-indicated conditions, or alternatively about 0.5 mg to about 7 g per patient per day. For example, the diseases and conditions described herein may be effectively treated by the administration of from about 0.01 to 50 mg of the compound per kilogram of body weight per day, or alternatively about 0.5 mg to about 3.5 g per patient per day.

A compound of the invention is typically combined with the carrier to produce a dosage form suitable for the particular patient being treated and the particular mode of administration. For example, a formulation intended for the oral administration to humans may contain from about 0.5 mg to about 5 g of the compound of the invention, compounded with an appropriate and convenient amount of carrier material which may vary from about 5 to about 95 percent of the total composition. Representative dosage forms will generally contain between from about 1 mg to about 500 mg of a compound of the invention, typically 25 mg, 50 mg, 100 mg, 200 mg, 300 mg, 400 mg, 500 mg, 600 mg, 800 mg, or 1000 mg.

It is understood that the specific dose level for any particular patient will depend upon a variety of factors including the age, body weight, general health, sex, diet, time of administration, route of administration, rate of excretion, drug combination and the severity of the particular disease undergoing therapy.

In this capacity, and as appreciated by those of skill in the art, therapy comprising administration of compounds of the present invention may include co-administration of one or more additional active agents. Classes of active agents include, but are not limited to β2-adrenergic agonists; anti-cholinergic agents; steroids; non-steroidal anti-inflammatory agents (NSAID's); mucolytic agents; and antibacterials.

β2-adrenergic agonists include, but are not limited to, metaproterenol, terbutaline, isoetharine, albuterol, and ritodrine, carbuterol, fenoterol, quinterenol, rimiterol, salmefamol, soterenol, and tretoquinol.

Anti-cholinergic agents include, but are not limited to, atropine, and iptratropium-bromide.

Mucolytic agents include, but are not limited to acetylcysteine and guattenesin.

Steroids include, but are not limited to, prednisone, beclomethasone, budesonide, solumedrol, triamcinolone, and methyl-prednisolone.

Non-steroidal anti-inflammatory agents include, but are not limited to aspirin, diflunisal, naphthylsalicylate, phenylbutazolone, oxyphenbutazolone, indomethacin, sulindac, mefenamic acid, meclofenamate sodium, tolmetin, ibuprofen, naproxen, fenoprofen and piroxicam.

Antibacterial agents include the broad classes of penicillins, cephalosporins and other beta-lactams, aminoglycosides, quinolones, macrolides, tetracyclines, sulfonamides, lincosamides and polymyxins. The penicillins include, but are not limited to penicillin G, penicillin V, methicillin, nafcillin, oxacillin, cloxacillin, dicloxacillin, floxacillin, ampicillin, ampicillin/sulbactam, amoxicillin, amoxicillin/clavulanate, hetacillin, cyclacillin, bacampicillin, carbenicillin, carbenicillin indanyl, ticarcillin, ticarcillin/clavulanate, azlocillin, mezlocillin, peperacillin, and mecillinam. The cephalosporins and other beta-lactams include, but are not limited to cephalothin, cephapirin, cephalexin, cephradine, cefazolin, cefadroxil, cefaclor, cefamandole, cefotetan, cefoxitin, ceruroxime, cefonicid, ceforadine, cefixime, cefotaxime, moxalactam, ceftizoxime, cetriaxome, ceftizoxime, cetriaxone, cephoperazone, ceftazidime, imipenem and aztreonam. The aminoglycosides include, but are not limited to streptomycin, gentamicin, tobramycin, amikacin, netilmicin, kanamycin and neomycin. The quinolones include, but are not limited to nalidixic acid, norfloxacin, enoxacin, ciprofloxacin, ofloxacin, sparfloxacin and temafloxacin. The macrolides include, but are not limited to erythomycin, spiramycin and azithromycin. The tetracyclines include, but are not limited to doxycycline, minocycline and tetracycline. The sulfonamides include, but are not limited to sulfanilamide, sulfamethoxazole, sulfacetamide, sulfadiazine, sulfisoxazole and co-trimoxazole (trimethoprim/sulfamethoxazole). The lincosamides include, but are not limited to clindamycin and lincomycin. The polymyxins (polypeptides) include, but are not limited to polymyxin B and colistin.

Where a second pharmaceutical is used in addition to a compound of the invention described herein, the two pharmaceuticals may be administered together in a single composition, separately at approximately the same time, or on separate dosing schedules. The important feature is that their dosing schedules comprise a treatment plan in which the dosing schedules overlap in time and thus are being followed concurrently.

Any suitable route of administration may be employed for providing the patient with an effective dosage (e.g., oral, sublingual, rectal, intravenous, epidural, intrethecal, subcutaneous, transcutaneous, intramuscular, intraperitoneal, intracutaneous, inhalation, transdermal, nasal spray or drop, and the like). While it is possible that, for use in therapy, compounds of the present invention may be administered as the pure chemicals without carriers, excipients and the like, as by inhalation of a fine powder via an insufflator, it is preferable to present the active ingredient as a pharmaceutical formulation. The invention thus further provides a pharmaceutical formulation comprising a compound of the present invention, together with one or more pharmaceutically acceptable carriers therefor and, optionally, other therapeutic and/or prophylactic ingredients. The carrier(s) must be ‘acceptable’ in the sense of being compatible with the other ingredients of the formulation and not deleterious to the recipient thereof, such as a human patient or domestic animal.

Pharmaceutical formulations include those suitable for oral or parenteral (including intramuscular, subcutaneous and intravenous) administration. Forms suitable for parenteral administration also include forms suitable for administration by inhalation or insufflation or for nasal, or topical (including buccal, rectal, vaginal and sublingual) administration. The formulations may, where appropriate, be conveniently presented in discrete unit dosage forms and may be prepared by any of the methods well known in the art of pharmacy. Such methods include the step of bringing into association a compound of the invention with liquid carriers, solid matrices, semi-solid carriers, finely divided solid carriers or combinations thereof, and then, if necessary, shaping the product into the desired delivery system.

Pharmaceutical formulations suitable for oral administration may be presented as discrete unit dosage forms such as hard or soft gelatin capsules, cachets or tablets each containing a predetermined amount of the active ingredient; as a powder or asganules; as a solution, a suspension or as an emulsion; or in a chewable base such as a synthetic resin or chicle for ingestion of the agent from a chewing gum. A compound of Formula I or Formula II may also be presented as a bolus, electuary or paste. Tablets and capsules for oral administration may contain conventional excipients such as binding agents, fillers, lubricants, disintegrants, or wetting agents. The tablets may be coated according to methods well known in the art, i.e., with enteric coatings.

Oral liquid preparations may be in the form of, for example, aqueous or oily suspensions, solutions, emulsions, syrups or elixirs, or may be presented as a dry product for constitution with water or other suitable vehicle before use. Such liquid preparations may contain conventional additives such as suspending agents, emulsifying agents, non-aqueous vehicles (which may include edible oils), or preservatives.

The compounds according to the invention may also be formulated for parenteral administration (e.g., by injection, for example, bolus injection or continuous infusion) and may be presented in unit dose form in ampules, pre-filled syringes, small volume infusion containers or in multi-dose containers with an added preservative. The compositions may take such forms as suspensions, solutions, or emulsions in oily or aqueous vehicles, and may contain formulatory agents such as suspending, stabilizing and/or dispersing agents. Alternatively, a compound of the invention may be in powder form, obtained by aseptic isolation of sterile solid or by lyophilization from solution, for constitution with a suitable vehicle, e.g., sterile, pyrogen-free water, before use.

For topical administration to the epidermis, the compounds may be formulated as ointments, creams or lotions, or as the active ingredient of a transdermal patch. Suitable transdermal delivery systems are disclosed, for example, in A. Fisher et al. (U.S. Pat. No. 4,788,603), or R. Bawa et al. (U.S. Pat. Nos. 4,931,279; 4,668,506 and 4,713,224). Ointments and creams may, for example, be formulated with an aqueous or oily base with the addition of suitable thickening and/or gelling agents. Lotions may be formulated with an aqueous or oily base and will in general also contain one or more emulsifying agents, stabilizing agents, dispersing agents, suspending agents, thickening agents, or coloring agents.

Formulations suitable for topical administration in the mouth include unit dosage forms such as lozenges comprising active ingredient in a flavored base, usually sucrose and acacia or tragacanth; pastilles comprising the active ingredient in an inert base such as gelatin and glycerin or sucrose and acacia; mucoadherent gels, and mouthwashes comprising a compound of the invention in a suitable liquid carrier.

When desired, the above-described formulations can be adapted to give sustained release of the active ingredient employed, e.g., by combination with certain hydrophilic polymer matrices, e.g., comprising natural gels, synthetic polymer gels or mixtures thereof. The polymer matrix can be coated onto, or used to form, a medical prosthesis, such as a stent, valve, shunt, gaft, or the like.

Pharmaceutical formulations suitable for rectal administration wherein the carrier is a solid are most preferably presented as unit dose suppositories. Suitable carriers include cocoa butter and other materials commonly used in the art, and the suppositories may be conveniently formed by admixture of a compound of the invention with the softened or melted carrier(s) followed by chilling and shaping in molds.

Formulations suitable for vaginal administration may be presented as pessaries, tampons, creams, gels, pastes, foams or sprays containing, in addition to a compound of the invention, such carriers as are known in the art to be appropriate.

For administration by inhalation, the compounds according to the invention are conveniently delivered from an insufflator, nebulizer or a pressurized pack or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount.

Alternatively, for administration by inhalation or insufflation, the compounds according to the invention may take the form of a dry powder composition, for example, a powder mix of the compound and a suitable powder base such as lactose or starch. The powder composition may be presented in unit dosage form in, for example, capsules or cartridges or, e.g., gelatin or blister packs from which the powder may be administered with the aid of an inhalator or insufflator.

For intra-nasal administration, the compounds of the invention may be administered via a liquid spray, such as via a plastic bottle atomizer. Typical of these are the Mistometer® (Wintrop) and the Medihaler® (Riker).

For topical administration to the eye, the compounds can be administered as drops, gels (U.S. Pat. No. 4,255,415), gums (see U.S. Pat. No. 4,136,177) or via a prolonged-release ocular insert.

The invention will now be described in greater detail by reference to the following non-limiting examples.

### EXAMPLES

### Example 1

### Preparation of (S)-tert-butyl-3-methyl-1-oxobutan-2-ylcarbamate (3)

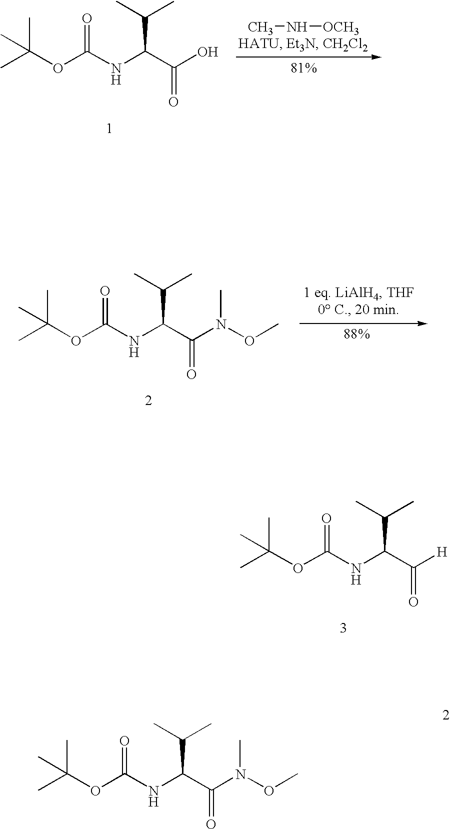

### (S)-[1-(Methoxy-methyl-carbamoyl)-2-methyl-propyl]-carbamic acid-tert-butyl ester (2)

To (S)-2-tert-butoxycarbonylamino-3-methyl-butyric acid (10 g, 46 mmol) [Novabiochem] in anhydrous methylene chloride (100 mL) at 0° C., was added triethylamine (7.0 mL, 51 mmol) and (2-(7-aza-1H-benzotriazole-1-yl)-1,1,3,3-tetramethyluronium hexafluorophosphate) [HATU] (19 g, 51 mmol). The resulting mixture was stirred vigorously for 20 minutes to obtain a homogeneous solution that contained a small amount of fine precipitate. A combination of N,O-dimethylhydroxylamine hydrochloride (5.4 g, 55 mmol) and triethylamine (7.6 mL, 55 mmol) was then added, and the resulting solution was warmed up to room temperature with stirring over three hours. The reaction was poured into a separatory funnel and the organic layer was washed in succession with aqueous 1 M HCl (2×75 mL), saturated NaHCO₃ (1×75 mL), and saturated NaCl (50 mL). The organic layer was dried over Na₂SO₄, filtered, and evaporated in vacuo. The resulting residue was subjected to silica gel chromatography using a solvent system of 1/1 hexanes/ethyl acetate to yield 9.7 g (37 mmol, 81%) of the title compound as a clear, colorless oil. ¹H NMR (300 MHz, CDCl₃) δ 5.13 (br d, 1H, J =9.0 Hz), 4.57 (m, 1H), 3.77 (s, 3H), 3.22 (s, 3H), 1.99 (dqq, 1H, J=6.6 Hz), 1.44 (s, 9H), 0.96 (d, J=6.9 Hz, 3H), 0.92 (d, J=6.9 Hz, 3H). ESI-MS m/z 261 (M+H).

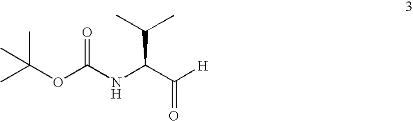

### (S)-(1-Formyl-2-methyl-propyl)-carbamic acid tert-butyl ester (3)

To (S)-[1-(methoxy-methyl-carbamoyl)-2-methyl-propyl]-carbamic acid-tert-butyl ester 2 (9.7 g, 37 mmol) in anhydrous tetrahydrofuran (100 mL) at 0° C., was added lithium aluminum hydride (1.4 g, 37 mmol) in portions over 25 minutes. The resulting solution was stirred for 15 minutes at 0° C. NaHSO₄ (4.5 g, 37 mmol) in H₂O (15 mL) was added dropwise over 5 minutes, and the resulting solution was poured into diethyl ether (200 mL). The organic layer was washed in succession with aqueous 1 M HCl (2×75 mL), saturated NaHCO₃ (1×75 mL), and saturated NaCl (1×50 mL). The organic layer was then dried over Na₂SO₄, filtered, and evaporated in vacuo to yield the title compound 6.6 g (33 mmol, 88%) as a clear, colorless oil. The aldehyde was used immediately in the next synthetic step without any further purification. ¹H NMR (300 MHz, CDCl₃) δ 9.62 (s, 1H), 5.09 (br s, 1H), 4.24 (br m, 1H), 2,28 (m, 1H), 1.45 (s, 9H), 1.03 (d, 3H, J=6.9 Hz), 0.92 (d, J=6.9 Hz, 3H).

### Example 2

### Preparation of H-Val-Pro-Val-Phenyl Oxadiazole HCl Salt (6)

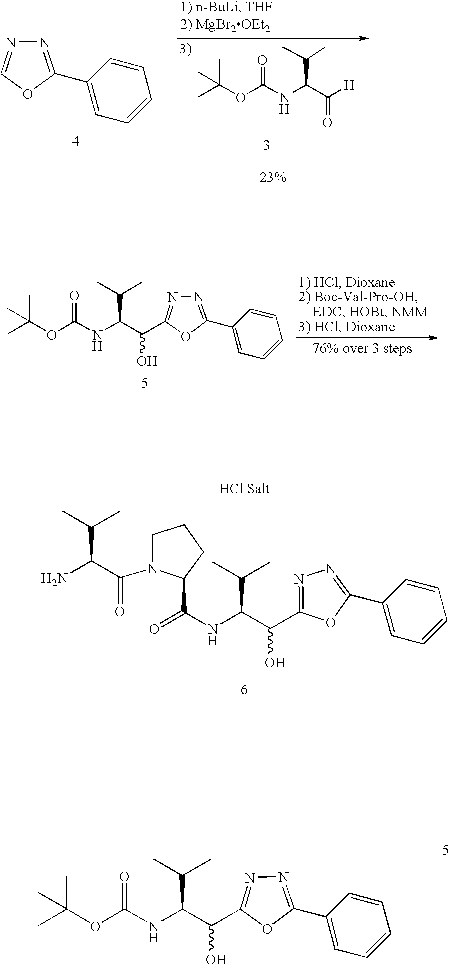

### (S)-tert-butyl-1-hydroxy-3-methyl-1-(5-phenyl-1,3,4-oxadiazol-2-yl)butan-2-ylcarbamate (5)

To a stirred solution of 2-phenyl-[1,3,4]oxadiazole 4 (5.5 g, 37 mmol) in anhydrous tetrahydrofuran (167 mL) under argon at −78° C. was added n-BuLi (15 mL of a 2.5 M solution in hexanes, 37 mmol) in a dropwise fashion. After stirring the resulting mixture for 90 minutes at −78° C., MgBr₂.OEt₂ (9.6 g, 37 mmol) was added. The reaction mixture was allowed to warm to —45° C., and allowed to stir at this temperature for 90 minutes. (1-Formyl-2-methyl-propyl)-carbamic acid tert-butyl ester 3 (8.0 g, 40 mmol) in tetrahydrofuran (52 mL) was added gradually, with the internal reaction temperature being kept below −35° C. The reaction mixture temperature was raised to −20° C. and the resulting mixture was stirred for 90 minutes. The reaction was then quenched with saturated NH₄Cl and extracted with ethyl acetate (100 mL). The organic layer was washed with aqueous saturated NaCl (50 mL), dried over Na₂SO₄, filtered, and evaporated in vacuo. The resulting residue was subjected to silica gel chromatography using a gradient of 20/1→1/1 hexanes/ethyl acetate to yield 2.0 g (5.8 mmol, 16%) of the title compound as a white foam. ¹H NMR (300 MHz, CDCl₃) δ 8.04 (m, 2H), 7.51 (m, 3H), 5.23-4.94 (m, 2H), 4.27-3.40 (m, 3H), 2.11 (m, 1H), 01.61-0.93 (m/z 348.3 (M+H).

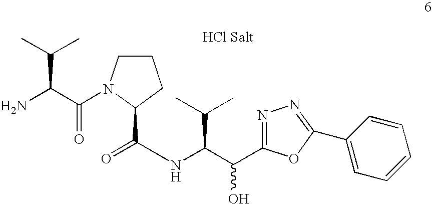

### (R)-1-((S)-2-amino-3-methylbutanoyl)-N-((S)-1-hydroxy-3-methyl-1-(5-phenyl-1,3,4-oxadiazol-2-yl)butan-2-yl)pyrrolidine-2-carboxamide hydrochloride salt (6)

A mixture (S)-tert-butyl-1-hydroxy-3-methyl-1-(5-phenyl-1,3,4-oxadiazol-2-yl)butan-2-ylcarbamate 5 (2.0 g, 5.8 mmol) and 4 N HCl in dioxane (25 mL, 100 mmol) diluted with dioxane (5 mL) was vigorously stirred at room temperature for 90 minutes. Concentration of the reaction mixture and solidification of the residue with diethyl ether, followed by azeotropic removal of water with toluene, gave 1.5 g (5.3 mmol, 94%) of the amino alcohol HCl salt. This material was added to a stirred mixture of (R)-1-((S)-2-(tert-butoxycarbonylamino)-3-methylbutanoyl)pyrrolidine-2-carboxylic acid (1.7 g, 5.5 mmol) and hydroxybenzotriazole hydrate (0.74 g, 5.5 mmol) in anhydrous dimethylformamide (15 mL) under argon at 0° C. EDC.HCl (1.0 g, 5.5 mmol) and N-methylmorpholine (0.60 mL, 5.5 mmol) were then added, and the reaction mixture was allowed to stir at room temperature for four hours. The reaction mixture was partitioned between ethyl acetate (100 mL) and saturated aqueous NH₄Cl (50 mL). The organic layer was washed with aqueous saturated NaHCO₃ (50 mL) and saturated NaCl (2×50 mL), followed by drying over Na₂SO₄, filtration, and evaporation in vacuo. The product was mixed with 4 N HCl in dioxane (25 mL, 100 mmol) diluted with dioxane (5 mL), and the resulting mixture was vigorously stirred at room temperature for two hours. Concentration of the reaction mixture and trituration of the residue with diethyl ether gave 2.1 g (4.4 mmol, 76%) of the title compound as a free-flowing white solid. ¹H NMR (300 MHz, CDCl₃) δ 8.43 (s, 1H), 8.18 (s, 3H), 7.96 (m, 2H), 7.46 (m, 3H), 5.32 (m, 1H), 4.80-3.40 (m, 5H), 2.16-1.19 (m, 7), 1.26-0.97 (m, 12H). ESI-MS m/z 444.6 (M+H).

### Example 3

### Preparation of H-Val-Pro-Val Amides (9), (13), and (16)

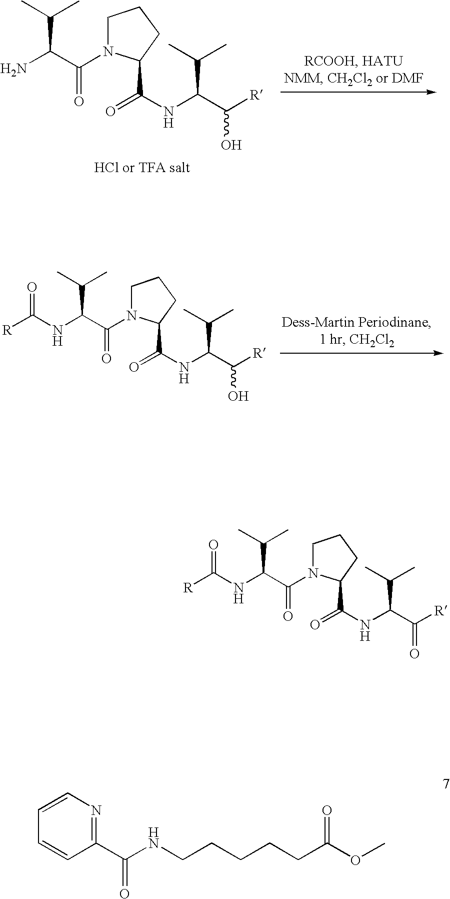

### 6-[(Pyridine-2-carbonyl)-amino]-hexanoic acid methyl ester (7)

To picolinic acid (0.52 g, 4.2 mmol) and 6-aminohexanoic acid methyl ester hydrochloride salt (0.78 g, 4.3 mmol) in anhydrous dimethylformamide under argon was added hydroxybenzotriazole hydrate (0.98 g, 6.5 mmol), N-methylmorpholine (1.9 mL, 17 mmol), and EDC.HCl (1.2 g, 6.5 mmol). The resulting mixture was stirred at room temperature overnight. The reaction mixture was partitioned between ethyl acetate (120 mL) and saturated aqueous NaHCO₃ (50 mL). The organic layer was washed with saturated aqueous NaHCO₃ (1×50 mL), water (2×50 mL), and saturated NaCl (1×50 mL), followed by drying over Na₂SO₄, filtration, and evaporation in vacuo. The resulting residue was purified via silica gel chromatography using 1/1 hexanes/ethyl acetate to yield 0.82 g (3.3 mmol, 77%) of the title compound as a colorless oil. ¹H NMR (300 MHz, CDCl₃) δ 8.52 (d, 1H, J=4.5 Hz), 8.18 (d, 1H, J=7.8 Hz), 8.05 (br s, 1H), 7.86 (t, 1H, J=7.8 Hz), 7.40 (t, 1H, J=4.8 Hz), 3.65 (s, 3H), 3.47 (t, 2H, J=6.0 Hz), 2.39 (t, 2H, J=6.0 Hz), 1.67 (m, 4H), 1.42 (m, 2H). ESI-MS m/z 251.0 (M+H).

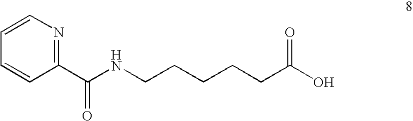

### 6-[(Pyridine-2-carbonyl)-amino]-hexanoic acid hydrochloride salt (8)

To 6-[(pyridine-2-carbonyl)-amino]-hexanoic acid methyl ester 7 (0.81 g, 3.3 mmol) in dioxane (10 mL) was added aqueous 1 M NaOH (6.5 mL, 6.5 mmol), and the resulting mixture was stirred at room temperature for one hour. The reaction was acidified to pH=1 with 0.5 N HCl, after which it was extracted with dichloromethane (3×25 mL). The combined dichloromethane extracts were washed with saturated aqueous NaCl (1×25 mL), followed by drying over Na₂SO₄, filtration, and evaporation in vacuo to yield the title compound 0.74 g (2.7 mmol, 84%). ¹H NMR (300 MHz, CDCl₃) δ 8.52 (d, 1H, J=4.8 Hz), 8.18 (d, 1H, J=7.8 Hz), 8.10 (br s, 1H), 7.83 (t, 1H, J=7.8 Hz), 7.40 (t, 1H, J=4.8 Hz), 3.42 (t, 2H, J=6.0 Hz), 2.37 (t, 2H, J=6.0 Hz), 1.67 (m, 4H), 1.42 (m, 2H). ESI-MS m/z 237.2 (M+H).

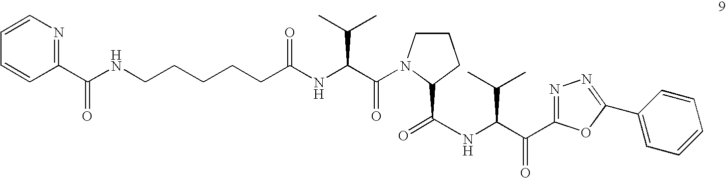

### N-(6-((S)-3-methyl-1-((R)-2-((S)-3-methyl-1-oxo-1-(5-phenyl-1,3,4-oxadiazol-2-yl)butan-2-ylcarbamoyl)pyrrolidin-1-yl)-1-oxobutan-2-ylamino)-6-oxohexyl) picolinamide (9)

To a stirred solution of 6-[(pyridine-2-carbonyl)-amino]-hexanoic acid hydrochloride salt 8 in methylene chloride (15 mL) was added (2-(7-aza-1H-benzotriazole-1-yl)-1,1,3,3-tetramethyluronium hexafluorophosphate) [HATU] (0.22 g, 0.58 mmol), followed by triethylamine (0.15 mL, 1.1 mmol). After the reaction mixture had stirred for five minutes, (R)-1-((S)-2-amino-3-methylbutanoyl)-N-((S)-1-hydroxy-3-methyl-1-(5-phenyl-1,3,4-oxadiazol-2-yl)butan-2-yl)pyrrolidine-2-carboxamide hydrochloride salt 6 (0.14 g, 0.30 mmol) was added, and the resulting mixture was stirred at room temperature for two hours. The reaction mixture was partitioned between methylene chloride (20 mL) and saturated aqueous NaCl (30 mL). The organic layer was washed with aqueous saturated NaHCO₃ (1×20 ML), 1 M HCl (1×20 mL) and saturated NaCl (1×50 mL), followed by drying over Na₂SO₄, filtration, and evaporation in vacuo. Purification of the residue via silica gel chromatography using 5/95 methanol/ethyl acetate gave the alcohol intermediate, which was dissolved in methylene chloride. To the resulting solution was added Dess-Martin Periodinane [1,1,1-tris(acetyloxy)-1,1-dihydro-1,2-benziodoxol-3-(1H)-one] (156 mg, 0.37 mmol), and the resulting mixture was stirred under argon at room temperature for one hour. The reaction mixture was partitioned between ethyl acetate (20 mL) and saturated aqueous NaCl (30 mL). The organic layer was dried over Na₂SO₄, filtered, and evaporated in vacuo. Purification of the residue via preparative HPLC yielded 42 mg (0.064 mmol, 43%) of the formic acid salt of the title compound as a white foam. ESI-MS m/z 660.8 (M+H).

### N-(6-((S)-3-methyl-1-((R)-2-((S)-3-methyl-1-oxo-1-(5-phenyl-1,3,4-oxadiazol-2-yl)butan-2-ylcarbamoyl)pyrrolidin-1-yl)-1-oxobutan-2-ylamino)-6-oxohexyl)picolinamide hydrochloride salt (9)

The HCl salt of the title compound was prepared from a methylene chloride solution of the free base by adding one equivalent of 1 M HCl in diethyl ether followed by concentration and drying to give a free-flowing white solid. ¹H NMR (300 MHz, CDCl₃) δ 8.58 (d, 1H, J=4.8 Hz), 8.48 (br s, 1H), 8.32 (d, 1H, J=7.8 Hz), 8.14 (m, 2H), 8.00 (t, 1H, J=7.2 Hz), 7.54 (m, 4H), 6.23 (d, 1H, J=8.4 Hz), 5.35 (m, 1H), 4.61 (m, 2H), 3.79 (m, 1H), 3.61 (m, 1H), 2.51-1.89 (m, 8H), 1.70 (m, 4H), 1.47 (m, 2H), 1.09-0.95 (m, 12H). ESI-MS m/z 660.6 (M+H).

### Example 4

### Preparation of H-Val-Pro-Val-t-Butyl Oxadiazole HCl Salt (12)

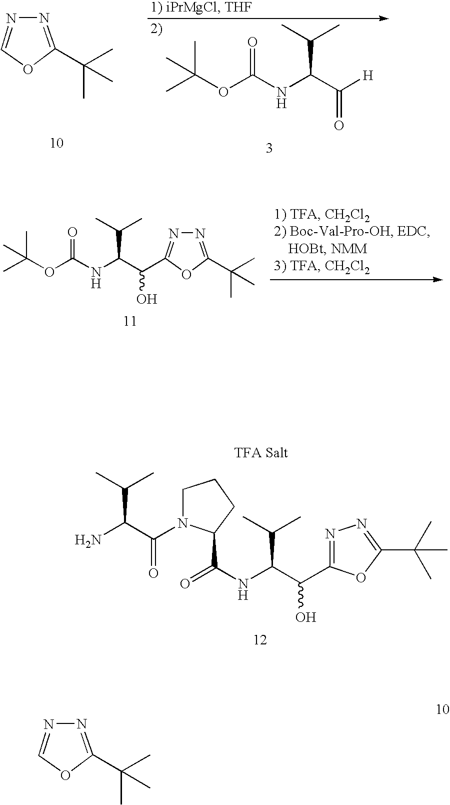

### 2-tert-Butyl-[1,3,4]oxadiazole (10)

To pivalohydrazide (1.6 g, 14 mmol) was added triethylorthoformate (3.5 mL, 21 mmol) and para-toluenesulfonic acid monohydrate (0.40 g, 2.1 mmol). The resulting mixture was heated at 120° C., removing ethanol by distillation. Distillation in vacuo of the remaining residue afforded 1.1 g (8.7 mmol, 63%) of the title compound as a yellow liquid. ¹H NMR (300 MHz, CDCl₃) δ 8.30 (s, 1H), 1.43 (s, 9H).

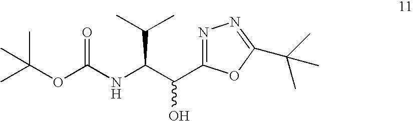

### (S)-tert-butyl-1-hydroxy-3-methyl-1-(5-phenyl-1,3,4-oxadiazol-2-yl)butan-2-ylcarbamate ( 11)

To a stirred solution of 2-tert-butyl-[1,3,4]oxadiazole 10 (0.2 g, 1.6 mmol) in anhydrous tetrahydrofuran (167 mL) under argon at −78° C. was added isopropyl magnesium chloride (2.4 mL of a 2.0 M solution in tetrahydrofuran, 4.8 mmol) in a dropwise fashion. (1-Formyl-2-methyl-propyl)-carbamic acid tert-butyl ester (0.28 g, 1.4 mmol) 3 in tetrahydrofuran (1 mL) was added dropwise. The reaction mixture temperature was raised to −20° C. and the resulting mixture was stirred for 90 minutes. The reaction was then quenched with saturated NH₄Cl and extracted with ethyl acetate (100 mL). The organic layer was washed with aqueous saturated NaCl (50 mL), dried over Na₂SO₄, filtered, and evaporated in vacuo. The resulting residue was carried on to the next step without further purification. ESI-MS m/z 328.4 (M+H).

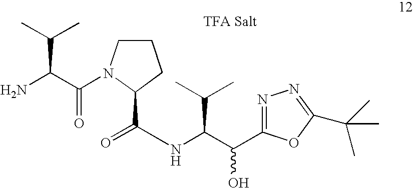

### (R)-1-((S)-2-amino-3-methylbutanoyl)-N-((S)-1-hydroxy-3-methyl-1-(5-phenyl-1,3,4-oxadiazol-2-yl)butan-2-yl)pyrrolidine-2-carboxamide trifluoroacetic acid salt (12)

A solution of (S)-tert-butyl-1-hydroxy-3-methyl-1-(5-phenyl-1,3,4-oxadiazol-2-yl)butan-2-ylcarbamate 11 (0.52 g, 1.6 mmol) in methylene chloride (2 mL) was treated with trifluoroacetic acid (2 mL). After stirring for one hour, the reaction was evaporated in vacuo and the residue was added to a stirred mixture of 1-(2-amino-3-methyl-butyryl)-pyrrolidine-2-carboxylic acid {1-[hydroxy-(5-phenyl-[1,3,4]oxadiazol-2-yl)-methyl]-2-methyl-propyl}-amide(0.50 g, 1.6 mmol) and hydroxybenzotriazole hydrate (0.25 g, 1.6 mmol) in anhydrous dimethylformamide (15 mL) under argon at 0° C. EDC.HCl (0.31 g, 1.6 mmol) and N-methylmorpholine (0.19 mL, 1.7 mmol) were then added, and the reaction mixture was allowed to stir at room temperature for four hours. The reaction mixture was partitioned between ethyl acetate (100 mL) and saturated aqueous NH₄Cl (50 mL). The organic layer was washed with aqueous saturated NaHCO₃ (50 mL) and saturated NaCl (2×50 mL), followed by drying over Na₂SO₄, filtration, and evaporation in vacuo. The product was mixed with trifluoroacetic acid (2 mL) in methylene chloride (2 mL), and the resulting mixture was vigorously stirred at room temperature for two hours. Concentration of the reaction mixture yielded the title compound as an oil. ESI-MS m/z 424.4 (M+H).

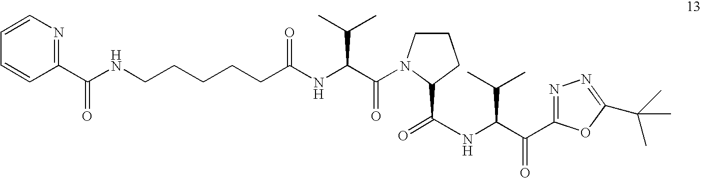

### N-(6-((S)-1-((R)-2-((S)-1-hydroxy-3-methyl-1-(5-phenyl-1,3,4-oxadiazol-2-yl)butan-2-ylcarbamoyl)pyrrolidin-1-yl)-3-methyl-1-oxobutan-2-ylamino)-6-oxohexyl)picolinamide (13)

To a stirred solution of 6-[(pyridine-2-carbonyl)-amino]-hexanoic acid hydrochloride salt 8 (0.40 g, 1. 5 mmol) in methylene chloride (10 mL) was added (2-(7-aza-1H-benzotriazole-1-yl)-1,1,3,3-tetramethyluronium hexafluorophosphate) [HATU] (1.1 g, 3.0 mmol). After the reaction mixture had stirred for five minutes, (R)-1-((S)-2-amino-3-methylbutanoyl)-N-((S)-1-hydroxy-3-methyl-1-(5-phenyl-1,3,4-oxadiazol-2-yl)butan-2-yl)pyrrolidine-2-carboxamide trifluoroacetic acid salt 12 (0.80 g, 1.5 mmol) in dimethylformamide (5 mL) was added, followed by 4-methyl morpholine (1.3 mL, 13.5 mmol). The resulting mixture was stirred at room temperature for two hours. The reaction mixture was partitioned between methylene chloride (20 mL) and saturated aqueous NaCl (30 mL). The organic layer was washed with aqueous saturated NaHCO₃ (1×20 mL), 1 M HCl (1×20 mL) and saturated NaCl (1×50 mL), followed by drying over Na₂SO₄, filtration, and evaporation in vacuo. To the resulting residue was added methylene chloride Dess-Martin Periodinane [1,1,1-tris(acetyloxy)-1,1-dihydro-1,2-benziodoxol-3-(1H)-one] (178 mg, 0.42 mmol), and the resulting mixture was stirred under argon at room temperature for one hour. The reaction mixture was partitioned between ethyl acetate (20 mL) and saturated aqueous NaCl (30 mL). The organic layer was dried over Na₂SO₄, filtered, and evaporated in vacuo. Purification of the residue via preparative HPLC yielded 26.5 mg of the formic acid salt of the title compound as an oil. ¹H NMR (300 MHz, CDCl₃) δ 9.43 (br s, 1H), 8.77 (d, J=6.0 Hz, 1H), 8.42 (m, 1H), 7.98 (m, 1H), 7.45 (m, 1H), 6.80 (m, 1H), 5.15 (m, 1H), 4.61 (m, 2H), 3.88-3.40 (m, 5H), 2.40-0.85 (m, 35H). ESI-MS m/z 640.4 (M+H).

### Example 5

### Preparation of (S)-3-amino-1,1,1-trifluoro-4-methylpentan-2-ol trifluoroacetic acid salt (14)

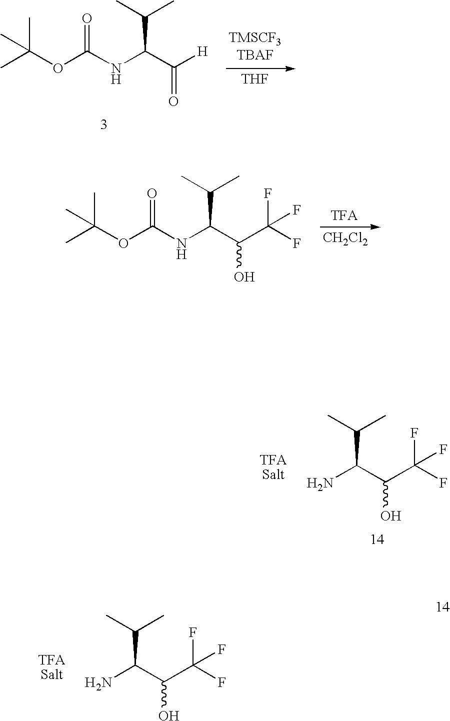

### (S)-3-amino-1,1,1-trifluoro-4-methylpentan-2-ol trifluoroacetic acid salt (14)

To (S)-(1-formyl-2-methyl-propyl)-carbamic acid tert-butyl ester 3 (0.3 g, 1.5 mmol) in anhydrous tetrahydrofuran (6 mL) at 0° C. was added trifluoromethyltrimethylsilane (0.44 mL, 3.0 mmol) in a dropwise fashion over 15 minutes. To the resulting solution was added tetrabutylammonium fluoride (2.3 mL of a 1 M solution in THF, 2.3 mmol) over 10 minutes. The resulting mixture was stirred at 0° C. for an additional 30 minutes, after which it was concentrated to dryness in vacuo. The residue was dissolved in dichloromethane (10 mL), to which trifluoroacetic acid was added. The resulting solution was stirred at room temperature for two hours, followed by concentration to dryness in vacuo to yield the title compound as a yellow oil.

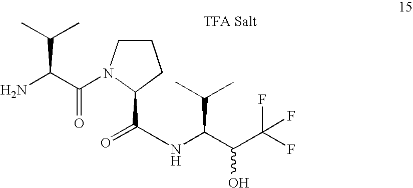

(R)-1-((S)-2-amino-3-methylbutanoyl)-N-((S)-1,1,1-trifluoro-2-hydroxy-4-methylpentan-3-yl)pyrrolidine-2-carboxamide trifluoroacetic acid salt (15)

A solution of (S)-3-amino-1,1,1-trifluoro-4-methylpentan-2-ol trifluoroacetic acid salt 14 (0.42 g, 1.5 mmol) in DMF (2 mL) was added to a stirred mixture of (R)-1-((S)-2-(tert-butoxycarbonylamino)-3-methylbutanoyl)pyrrolidine-2-carboxylic acid (0.34 g, 1.1 mmol) and hydroxybenzotriazole hydrate (0.25 g, 1.6 mmol) in anhydrous dimethylformamide (15 mL) under argon at 0° C. EDC.HCl (0.32 g, 1.7 mmol) and N-methylmorpholine (0.60 mL, 5.5 mmol) were then added, and the reaction mixture was allowed to stir at room temperature overnight. The reaction mixture was partitioned between ethyl acetate (100 mL) and saturated aqueous NH₄Cl (50 mL). The organic layer was washed with aqueous saturated NaHCO₃ (50 mL) and saturated NaCl (2×50 mL), followed by drying over Na₂SO₄, filtration, and evaporation in vacuo. The product was mixed with trifluoroacetic acid (2 mL) in methylene chloride (2 mL), and the resulting mixture was vigorously stirred at room temperature for two hours. Concentration of the reaction mixture yielded the title compound as an oil. ESI-MS m/z 368.4 (M+H).

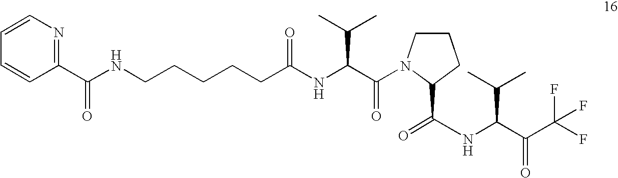

### N-(6-((S)-3-methyl-1-oxo-1-((R)-2-((S)-1,1,1-trifluoro-4-methyl-2-oxopentan-2-ylcarbamoyl)pyrrolidin-1-yl)butan-2-ylamino)-6-oxohexyl)picolinamide (16)

To a stirred solution of 6-[(pyridine-2-carbonyl)-amino]-hexanoic acid hydrochloride salt 8 (0.32 g, 1.2 mmol) in methylene chloride (10 mL) was added (2-(7-aza-1H-benzotriazole-1-yl)-1,1,3,3-tetramethyluronium hexafluorophosphate) [HATU] (0.90 g, 2.4 mmol). After the reaction mixture had stirred for five minutes, (R)-1-((S)-2-amino-3-methylbutanoyl)-N-((S)-1,1,1-trifluoro-2-hydroxy-4-methylpentan-3-yl) pyrrolidine-2-carboxamide trifluoroacetic acid salt 15 (0.44 g, 1.2 mmol) in dimethylformamide (5 mL) was added, followed by 4-methyl morpholine (1.3 mL, 13.5 mmol). The resulting mixture was stirred at room temperature for two hours. The reaction mixture was partitioned between methylene chloride (20 mL) and saturated aqueous NaCl (30 mL). The organic layer was washed with aqueous saturated NaHCO₃ (1×20 mL), 1 M HCl (1×20 mL) and saturated NaCl (1×50 mL), followed by drying over Na₂SO₄, filtration, and evaporation in vacuo. To a methylene chloride solution (10 mL) of 0.074 mmol of the resulting material was added Dess-Martin Periodinane [1,1,1-tris(acetyloxy)-1,1-dihydro-1,2-benziodoxol-3-(1H)-one] (63 mg, 0.15 mmol), and the resulting mixture was stirred under argon at room temperature for one hour. The reaction mixture was partitioned between ethyl acetate (20 mL) and saturated aqueous NaCl (30 mL). The organic layer was dried over Na₂SO₄, filtered, and evaporated in vacuo. Purification of the residue via preparative HPLC yielded 20.0 mg of the formic acid salt of the title compound as an oil. 1H NMR (300 MHz, CDCl3) δ 9.33 (br s, 1H), 8.75 (d, J=6.0 Hz, 1H), 8.63 (d, 1H, J=7.5 Hz), 8.32 (m, 1H), 8.02 (d, 1H, J=7.2 Hz), 7.86 (m, 1H), 6.43 (d, 1H, J=8.7 Hz), 4.83 (m, 1H), 4.61 (m, 2H), 3.88 (m, 1H), 3.67-3.50 (m, 4H), 2.37-2.27 (m, 3H), 2.06 (m, 4H), 1.73 (m, 4H), 1.46 (m, 2H), 1.10-0.92 (m, 12H). ESI-MS m/z 584.2 (M+H).

### Example 6

### Measurement of Enzyme and Inhibitory Activity

The assays for measurement of PR3 and HNE enzyme and inhibitory activity were performed generally as described by Coeschott et al., Proc. Nat. Acad. Sci. USA, 96: 6261-6266, (1999). Kinetic characterization was performed generally by the techniques of Wieczorek et al., Archives of Biochem. and BioPhys., 367: 193-201 (1999). Enzymes PR3 and HNE (HLE) were purchased from Athens Research and Technology (Athens, Ga.) or Elastin Products (Owensville, Mich.).

The substrate used for HNE was MeO-Suc-Ala-Ala-Pro-Val-pNA, in 0.05 M sodium phosphate/0.1 M NaCl, pH 7.6, containing 0.001% Triton X-100 and 5% DMSO. The substrate used for PR3 was Boc-Ala-Pro-Nva-4-chloro-SBzl in 0.05 M potassium phosphate/0.1 M KCl, pH 7.6, containing 0.001% Triton X-100, and 10% DMSO. The cleavage of the thiobenzyl ester was detected with 250 μM DTNG (5′,5′-dithiobis-2-nitrobenzoic acid).

Certain considerations were made when determining kinetics of HNE inhibitors.

(A) Assays for HNE Concentration

The concentration of active site of HNE was determined by titration with N-benzyloxycarbonyl-Ala-Ala-Pro-azaAla p-nitrophenyl ester or an alternative method. For the alternative method, the concentration of the active center of a HNE solution was first titrated with the above p-nitrophenyl ester, this HNE solution was then used to measure the active center concentration of an eglin c solution. Aliquots of the eglin c solution were stored at −70° C. and employed as a second standard to assay the activity of any newly prepared HNE solutions. No change in the activity of eglin c could be detected over a 3 year time interval as long as the solution was stored at −70° C. An important factor which influences the HNE assay is the method of dissolution of lyophilized enyme: if HNE is obtained in the lyophilized state, the protease solution should be dissolved, aliquoted, and stored at −20° C. for at least 48 hours prior to assay, so that the dried protein can be completely hydrated. Freshly dissolved enzyme which has not been stored for 48 hours may have proteolytic activity which is 10%-20% less that of stored enzyme.

By using the procedure described above, the HNE preparations from Athens Research and Technology, Inc, were about 50-60% active, while the fraction of active enzyme in HNE preparations from Elastin Products was typically less than 50%.

Since Ki for CE2072 is in the subnanomolar range, this compound can be considered to be a tight binding inhibitor of HLE. Consequently, it is not always easy to achieve conditions in which the initial concentration of free inhibitor, [I]o, is much larger than the initial concentration of free enzyme, [E]o, i.e. the condition of [I]o≧10 [E]o is not easily fulfilled. Rather, unless a high inhibitor concentration is employed (resulting in virtually total inhibition of HLE) or the enzyme concentration is maintained very low so that proportionately low inhibitor concentrations can also be used, the enzyme will bind a substantial fraction of CE2072, thereby changing the concentration of inhibitor, [I]. In these cases, the accuracy of determination of the starting enzyme concentration, [E]o, becomes very important for calculating the various kinetic constants, such as kon, koff and Ki. Indeed the two equations in the Wieczorek paper,

vₛ=vₒ/(1+[I]/IC₅₀)

and

[P]=[P]ₒ+vₛt−(vₛ−vₒ)(1−e^{-kt})/k

can only be employed when the condition of [I]ₒ≧10 [E]ₒ is fulfilled. In those cases in which the condition of [I]ₒ≧10 [E]ₒ is not met, the experimental data is analyzed using the Henderson equations according to the classic procedure (Henderson, P. J. F. Biochem. J. 127: 321, 1972) or with equations derived for slow-binding inhibition (Ying, Q-L. et al, Biochemistry 33: 5445, 1994). Both of these latter two approaches remain valid over a broad range of values for [I]ₒ and [E]ₒ.

(B) Assays for Substrate Concentration

The substrate MeO-Suc-Ala-Ala-Val p-nitroanilide was dissolved in DMSO, and prepared as a stock solution of millimolar concentration. Aliquots of this stock solution were diluted into distilled water to give concentrations of DMSO of less than 5%, and the concentrations of substrate in the diluted solutions were determined by measuring the absorbance at 316 nm, assuming an extinction coefficient, ε316 nm=12800 M⁻¹ cm⁻¹ for peptide p-nitroanilides (Friberger, P., Scand. J. Clin. Lab. Invest. 42, Suppl. 162: 1, 1982. Friberger examined the spectral properties of dozens of solutions of peptide p-nitroanilides of various lengths and amino acid compositions and reported that the values of their extinction coefficients, ε316 nm, were within a range from 12700 to 12800 M⁻¹ cm⁻¹).

By using this procedure, a solution of MeO-Suc-Ala-Ala-Val p-nitroanilide of a specific target concentration prepared by weighing the substrate, the actual concentration of the solute as determined by spectral measurements was found approximately 20% lower than that predicted from gravimetric measurements. Both classic and slow-binding methods of determining kinetic constants employ the concentration of substrate to calculate Ki. The accuracy of the method for establishing substrate concentration is also important for the determination of kinetic rate constants.

### (C) Determination of Km

The value of Km for the substrate MeO-Suc-Ala-Ala-Pro-Val p-nitroanilide binding to HNE was determined using the classic method based on the Michaelis-Menten equation. The value of Km used for the kinetic calculation was an average from three determinations. Each determination was based on a dataset of at least 7 points spanning 7 different concentrations of the substrate. The substrate concentrations varied from 0.2 Km to 20 Km.

In the literature, the value of Km for this substrate reported by different laboratories varies from 60 to 180 μM. The value reported by the laboratory of Dr. James Powers, who first synthesized and described the substrate, is 140 μM (Nakajima, K., et al, J. Biol. Chem. 254: 4027, 1979). The value of Km in DPBS buffer containing 10% DMSO, 0.1% Triton X-100, pH 7.2, was determined to be 97 μM, while in the pH 7.6 buffer of Wieczorek et al., the value was determined to be 103 μM.

Solubility of test compounds was measured by adding small volumes of DMSO stock solutions (final concentration 2% DMSO) to 0.1 M PBS (pH 7.4) and incubating for 2 hours at 37° C. Subsequently, the samples were filtered and the concentration of test compounds was determined by LC-MS.

Protein binding of test compounds in dog plasma was assessed by performing equilibrium dialysis with plasma containing test compound (10 μM) against 0.1 M PBS (pH 7.4). Following incubation (6 hours at 37° C.), the parent compound was measured in both plasma and buffer compartments by LC-MS and the percentage of compound bound to plasma proteins determined. The data from this study indicated that AI-158, AI-167 and AI-168 all exhibit moderate protein binding in dog plasma (92%, 81% and 83% bound, respectively). Generally it is believed that only the unbound drug is available for pharmacological action.

Plasma stability of test compounds was accessed by incubating with dog plasma (37° C. at 1 μM) for 2 hours. Aliquots were taken at pre-defined times and the disappearance of parent compound was monitored by LC-MS. The results of this study showed all three compounds to be stable in dog plasma (t½>100 minutes) and not susceptible to plasma enzyme hydrolysis.

TABLE 1
Inhibition Constants (Ki) and solubility data for CE-2072
and Select Compounds.

| | | | | Plasma | |
|---|---|---|---|---|---|
| | | | | Protein | Plasma |
| | Kᵢ (NE) | Kᵢ (PR3) | Solubility | Binding | Stability |
| Compound | nM | nM | (PBS, μM) | (% free) | (t_{1/2} minutes) |
| CE-2072 | 0.24 | 24 | <10 | 1.0 | >100 |
| AI-158 | 0.22 | 8.4 | 69.0 | 8.0 | >100 |
| AI-167 | — | — | 164.0 | 19.0 | >100 |
| AI-168 | 0.20 | 3.9 | 179.0 | 17.0 | >100 |

### Example 7

### Identification of Metabolites of AI-168

Previous results obtained in human liver microsomes, together with hamster and dog pharmacokinetic (PK) studies, suggested that AI-168 may be rapidly metabolized. Therefore, a study was performed to identify the metabolites of AI-168 formed in human liver microsomes to address the potential metabolic liability of this molecule.

The compound AI-168 (200 μM ) was incubated with human liver microsomes (0.5 mg/mL), and NADPH (1 mM), in 0.1 M phosphate buffer (pH 7.4) at 37° C. for 0 and 30 minutes. The reaction was terminated by addition of acetonitrile. The samples were centrifuged and the supernatant fraction analysed by LC-MS/MS. The presence of putative metabolites was monitored by mass spectrometry using full scan (100-720 amu) in positive ion mode. After the identification of potential metabolites, daughter ion scan mass chromatograms were generated for both parent compound and the metabolites. Fragmentation patterns for parent and metabolites were studied in order to try and locate the sites of metabolism.

An Applied Biosystems API 2000 (S/N: N0460310, Applied Biosystems, 850 Lincoln Centre Drive, Foster City, Calif.) using full scan was used for this study. A multiple reaction monitoring (MRM) method was developed for AI-168 using Analyst software from Applied Biosystems. The AI-168 metabolite supernatant was run on a Phenomenex Gemini 5 uM C18 110 50×4.6 mm column at 1.0 mL/min. Mobile phase A was 0.01% formic acid in water and mobile phase B was 0.01% formic acid in acetonitrile. The gradient profile was run from 5% B at 0 min to 95% B in 4 min.

Three putative metabolites of AI-168 were found in the 30 minute microsomal incubation supernatants, the molecular weights of these are shown in Table 2; the structures and proposed oxidative metabolic pathways to the metabolites are shown in FIG. 6.

TABLE 2
Proposed Metabolites.

| Metabolite | | Retention Time | |
|---|---|---|---|
| No. | MW | Minutes | Proposed Metabolite |
| 1 | 655.4 (Parent + 16) | 3.61 | Hydroxylation |
| 2 | 655.4 (Parent + 16) | 3.61 | Hydroxylation |
| 3 | 533.3 (Parent − 106) | 3.74 | C—N cleavage |

Two of the metabolites appear to be resultant from hydroxylations of AI-168; one “left” of the central amide, possibly on the pyridine ring, and one “right” of central amide group, possibly on the t-butyl or isopropyl group. The third metabolite represents cleavage of the molecule at “left” end of the alkyl chain, with formation of an aldehyde terminus. This may be a secondary step following hydroxylation at this carbon atom. A product of similar mass would also be formed if “amidase” hydrolysis occurred, resulting in a primary amine terminus. Proposed oxidative metabolic pathways are shown in FIG. 6.

Metabolite 1 (retention time 3.61 minutes) had a molecular weight of 655.4 amu, 16 amu greater than parent and indicative of oxidation. The proposed structure of metabolite 1 is shown in FIG. 6. The fragmentation pattern of this molecule was similar to parent with the exception of the ion at m/z 334.2 which is an increase of 16 amu from the ion of m/z 318.1 seen in the parent mass spectrum. This ion was assigned to a fragment containing the pyridine ring and it was proposed that metabolite 1 represents hydroxylation of this ring. The proposed structure is consistent with StarDrop (BioFocusDPI, San Diego, Calif.) prediction that this position on the ring is the most vulnerable to oxidation by cytochrome P450 enzymes.

Metabolite 2 (retention time 3.61 minutes) also had a mass of 655.4 amu, 16 amu greater than parent and indicative of oxidation, usually hydroxylation. The proposed structure of metabolite 2 is shown in FIG. 6. The fragmentation pattern of this metabolite was also similar to parent except for an ion of m/z 339.1, 16 amu greater than the corresponding ion from parent molecule. It was proposed that metabolite 2 was oxidation at either the t-butyl or isopropyl group.

Metabolite 3 (retention time 3.74 minutes) had a molecular weight of 533.5 amu (parent −106 amu) and the proposed structure is shown in FIG. 6. The fragmentation pattern showed an ion at m/z 323.3 as did the parent molecule. It was proposed that the parent molecule is hydroxylated next to the amide near the heterocyclic ring, forming an unstable molecule that undergoes a unimolecular reaction to form the aldehyde. This proposed metabolite structure is consistent with the StarDrop prediction that this site is the most vulnerable on the molecule to cytochrome P450 enzymes.

All references cited herein, including journal articles or abstracts, published or corresponding U.S. or foreign patent applications, issued U.S. or foreign patents, or any other references, are entirely incorporated by reference herein, including all data, tables, figures, and text presented in the cited references. Additionally, the entire contents of the references cited within the references cited herein are also entirely incorporated by reference.

Reference to known method steps, conventional methods steps, known methods or conventional methods is not in any way an admission that any aspect, description or embodiment of the present invention is disclosed, taught or suggested in the relevant art.

The foregoing description of the specific embodiments will so fully reveal the general nature of the invention that others can, by applying knowledge within the skill of the art (including the contents of the references cited herein), readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance presented herein, in combination with the knowledge of one of ordinary skill in the art.

We claim:

1. A compound of the formula (I): 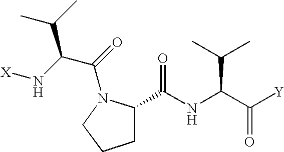
where X is R₁—(CR₃R₄)ₙOC(O)—; R₁—(CR₃R₄)ₙC(O)—; R₁—C(O)NH(CR₃R₄)ₙOC(O);
R₁—C(O)NH(CR₃R₄)ₙC(O)—; R₁—C(O)(CR₃R₄)ₙOC(O)—; or R₁—C(O)(CR₃R₄)ₙC(O);
where R₁ is optionally substituted C₅₋₁₀ aryl or heteroaryl; OH or NH₂; where R₃ and R₄ are independently H or methyl; and
n is 0 to 6; and
Y is —CF₃ or one of 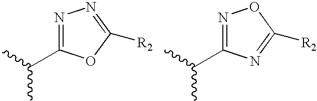
where R₂ is C₁₋₈ alkyl optionally substituted with halo or —OH; or —(CR₆R₇)ₚ—C₅₋₆ aryl optionally substituted with halo, —OH, C₁₋₈ alkyl, C₁₋₈ haloalkyl, —(CH₂)ₘC(O)NH₂ or —(CH₂)ₘOCH₃; where R₆ and R₇ are independently H or methyl; m is 0 to 4, and p is 0 or 1 or a pharmaceutically acceptable salt, ester, metabolite or prodrug thereof.

2. The compound of claim 1 wherein R₁ is 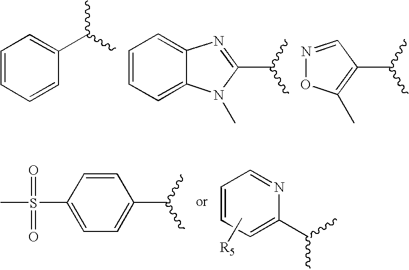
where R₅ is H, halo, OH or NR₃R₄.

3. The compound of claim 1 wherein X is: 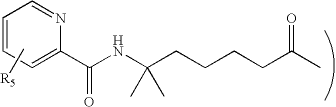
where R₅ is H, halo, or OH.

4. The compound of claim 1 wherein R₂ is

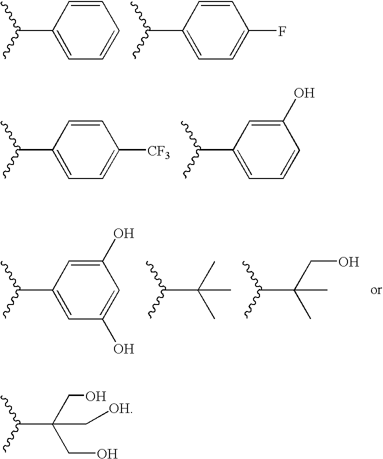

5. The compound of claim 1 wherein R₂ is —C(CH₃)₂—C₅₋₆ aryl substituted with —(CH₂)ₘC(O)NH₂ or —(CH₂)ₘOCH₃.

6. The compound of claim 1:

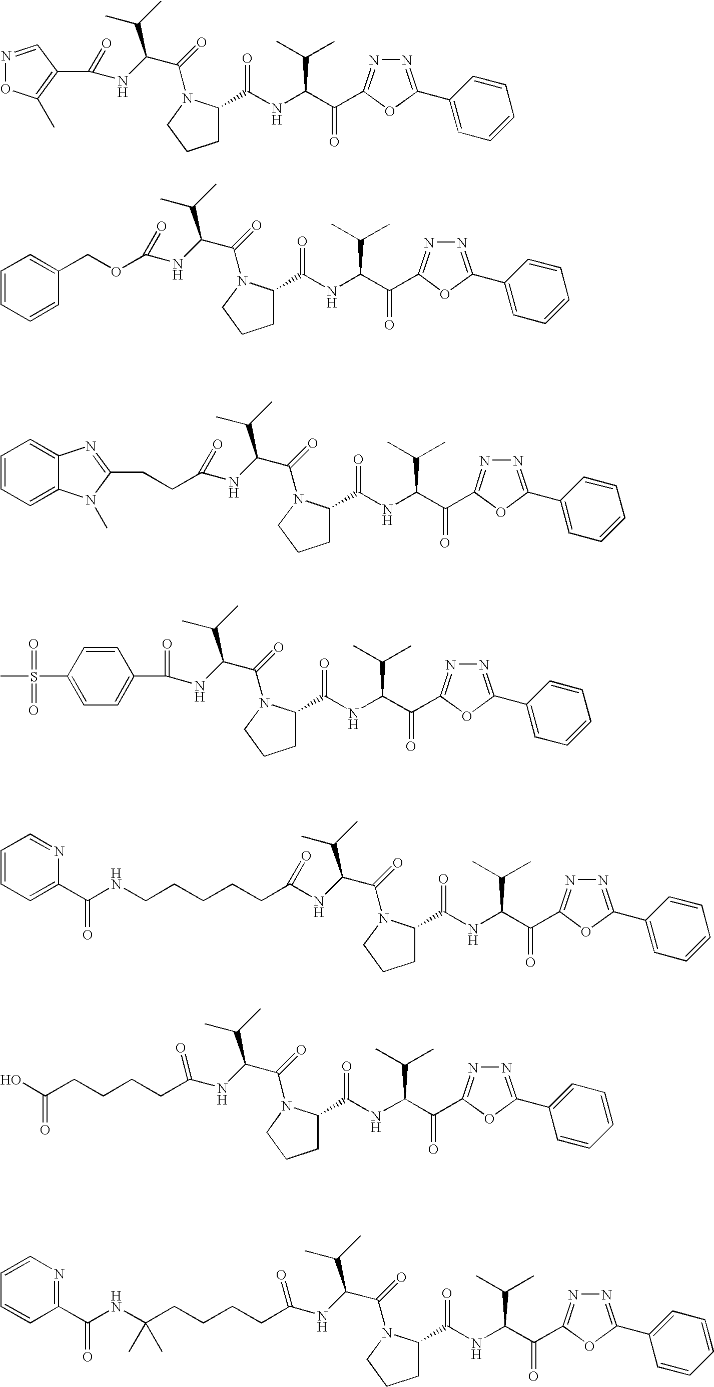

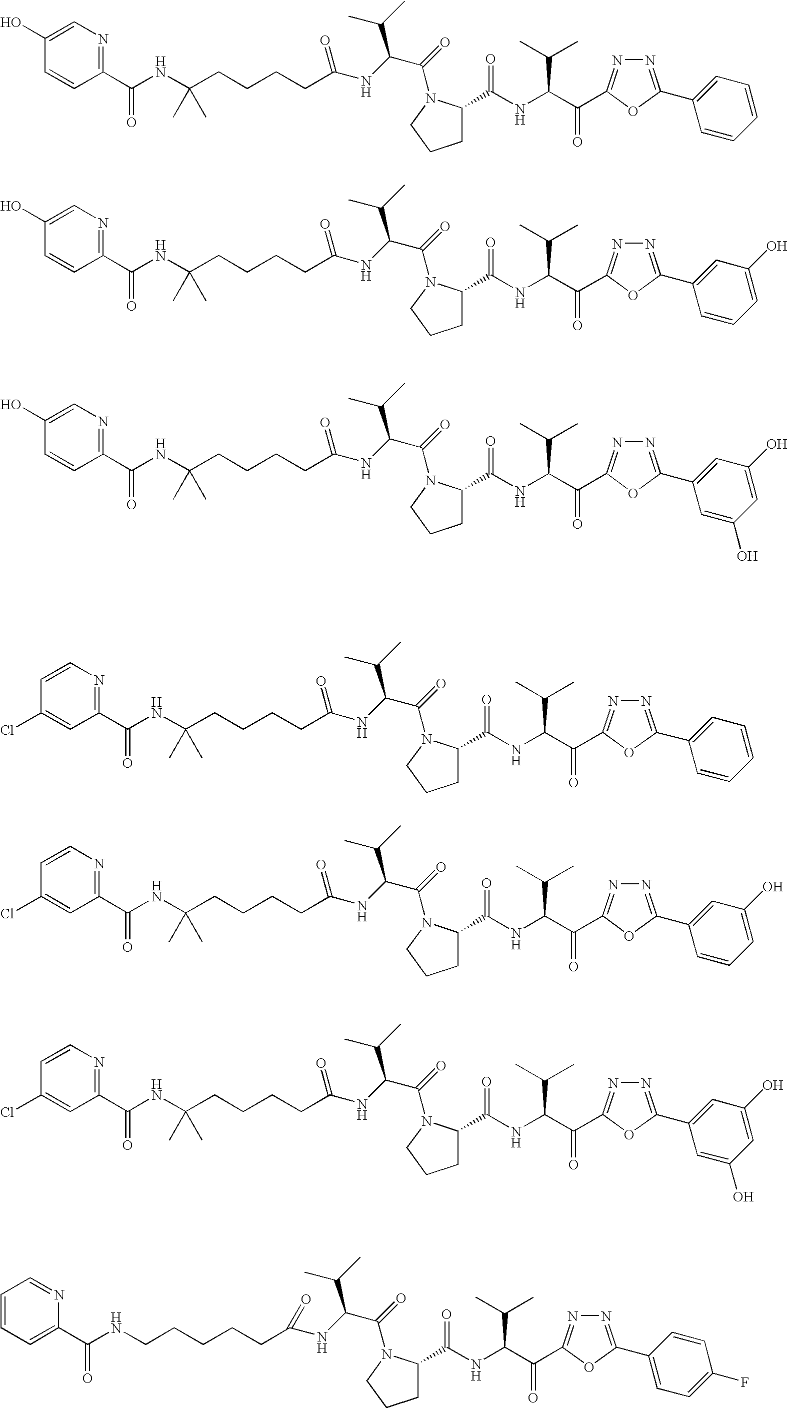

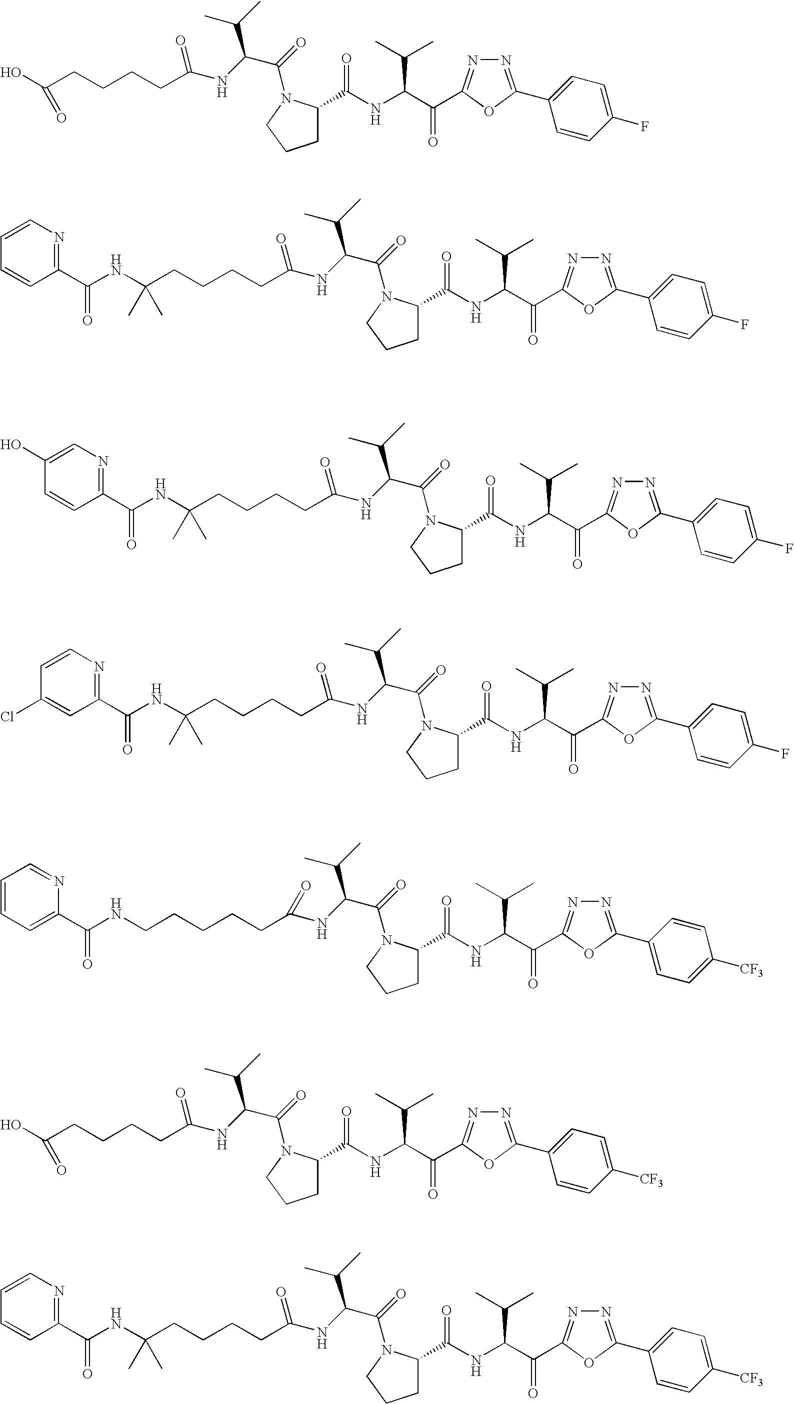

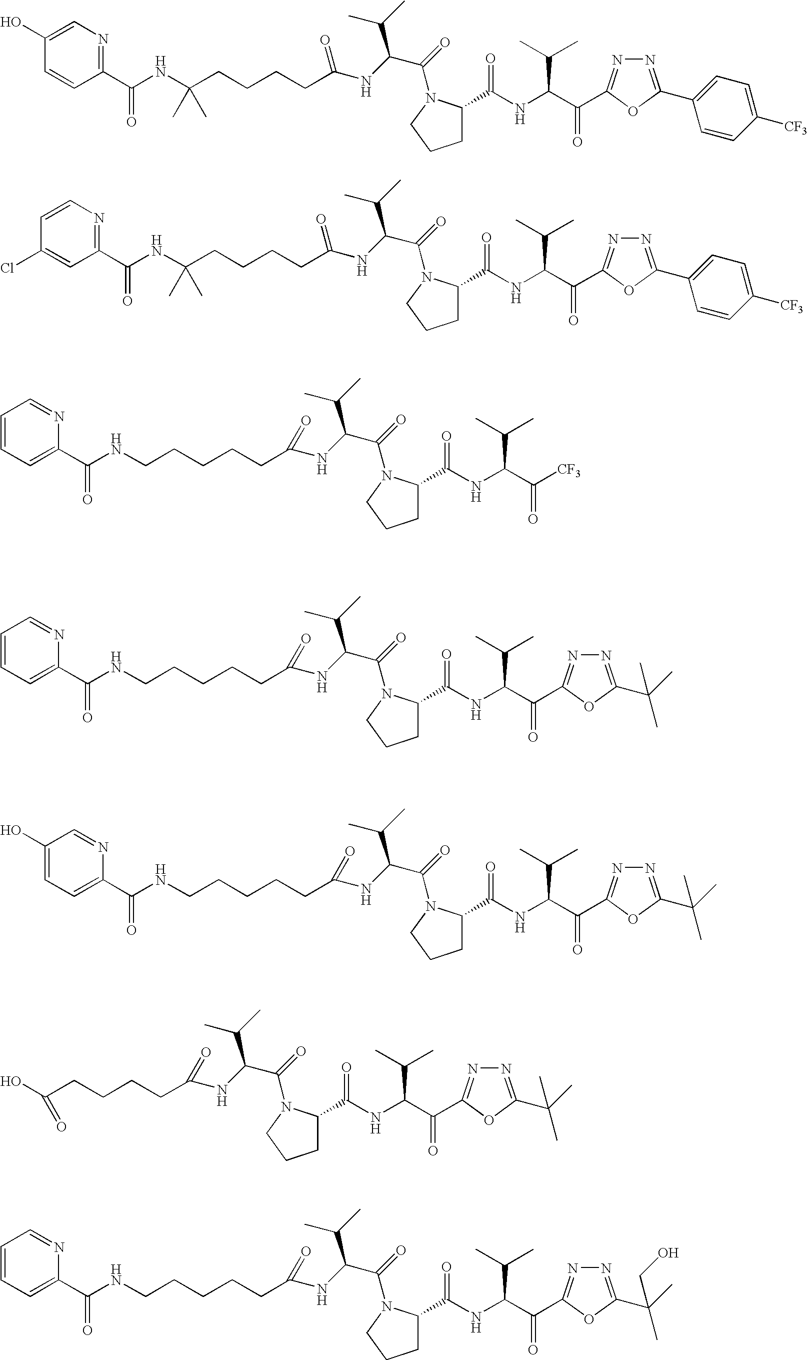

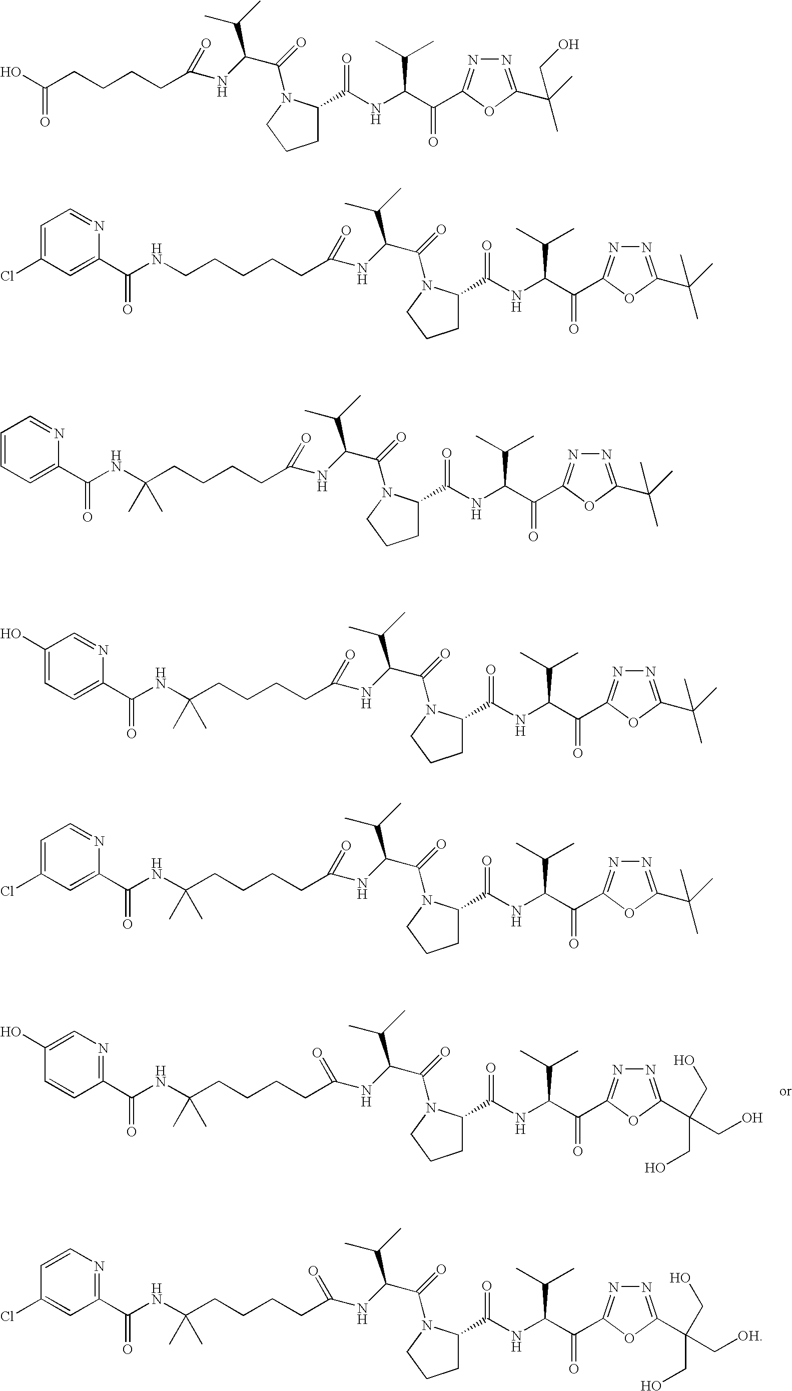

7. The compound of claim 6:

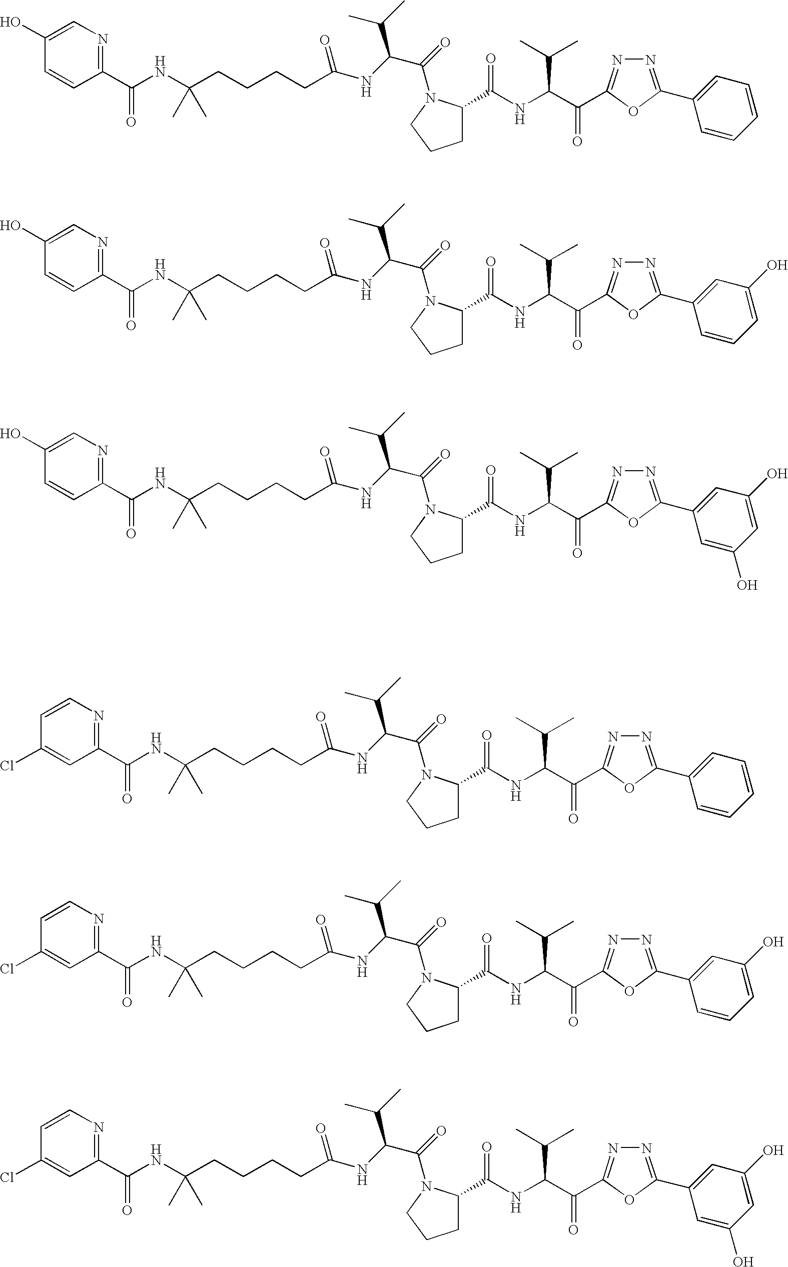

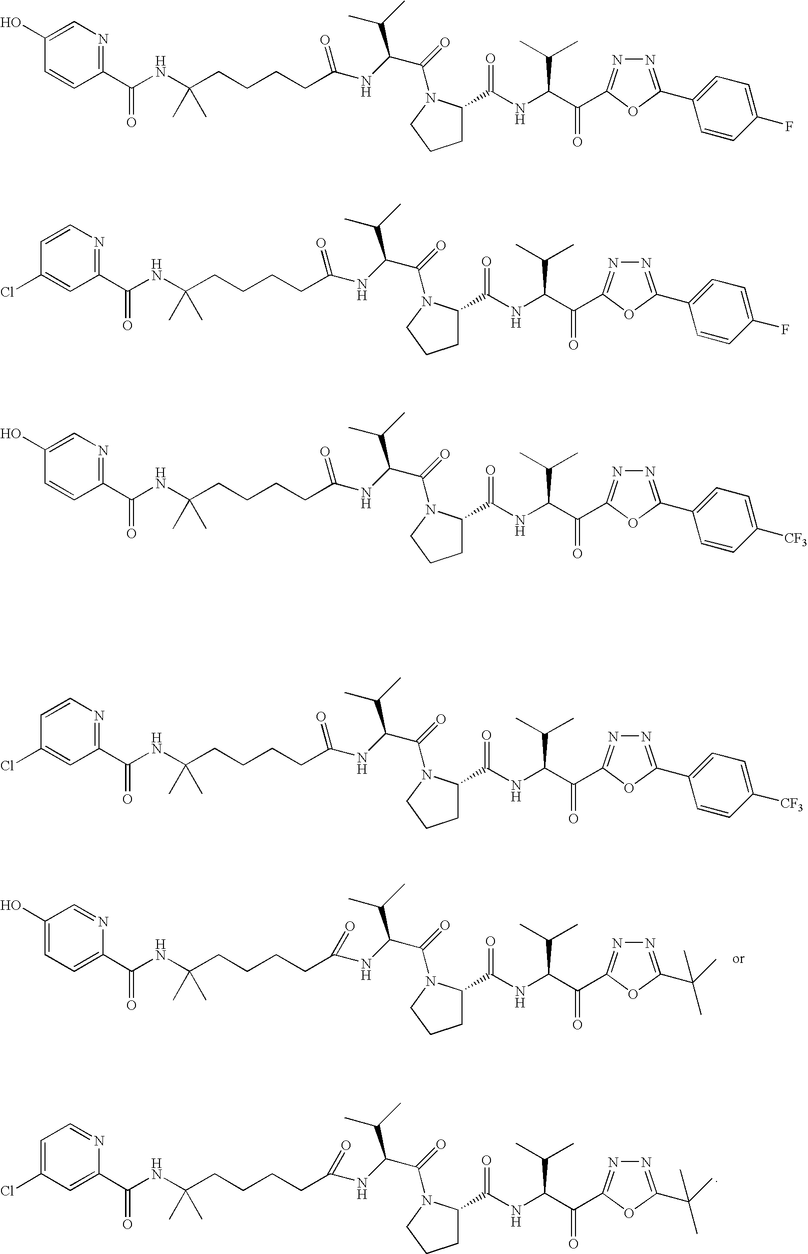

8. The compound of claim 7:

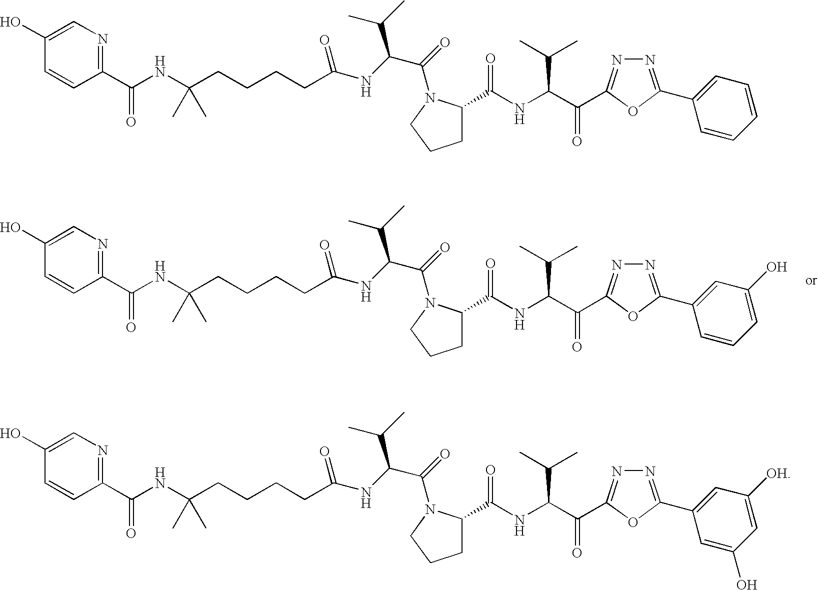

9. The compound of claim 7:

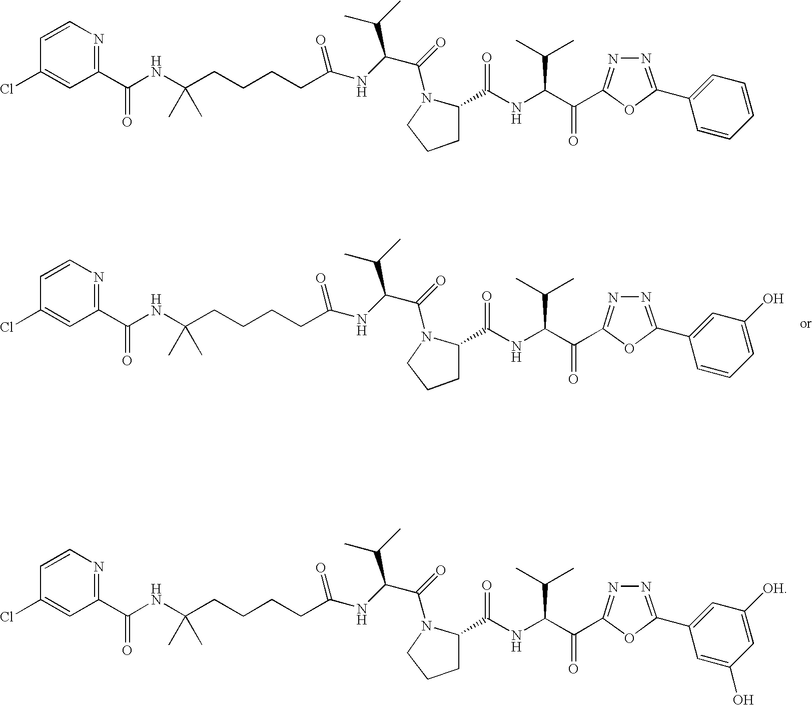

10. The compound of claim 7:

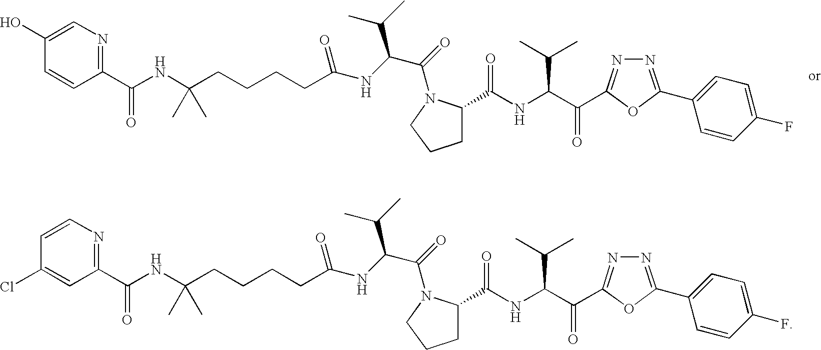

11. The compound of claim 7:

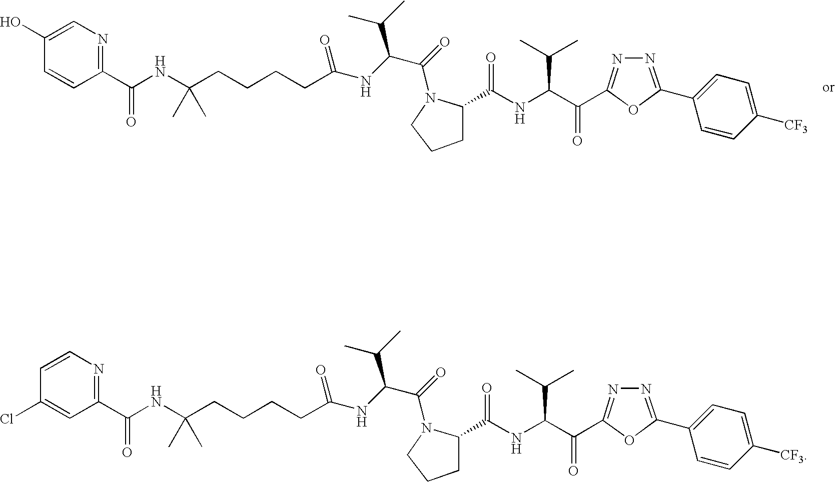

12. The compound of claim 7:

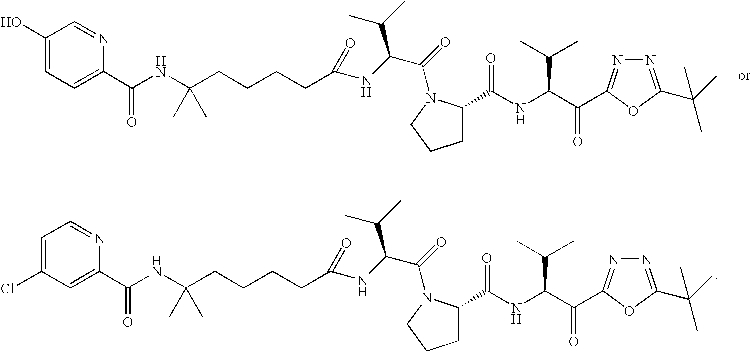

13. The compound of claim 6:

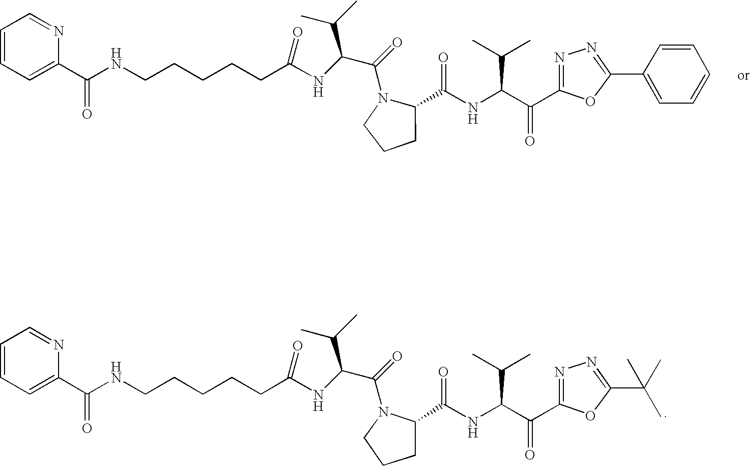

14. A pharmaceutical composition for the inhibition of HNE and PR3 comprising a therapeutically effective amount of a compound of claim 1 and a pharmaceutically acceptable carrier.

15. The pharmaceutical composition of claim 14, wherein the compound of claim 1 is:

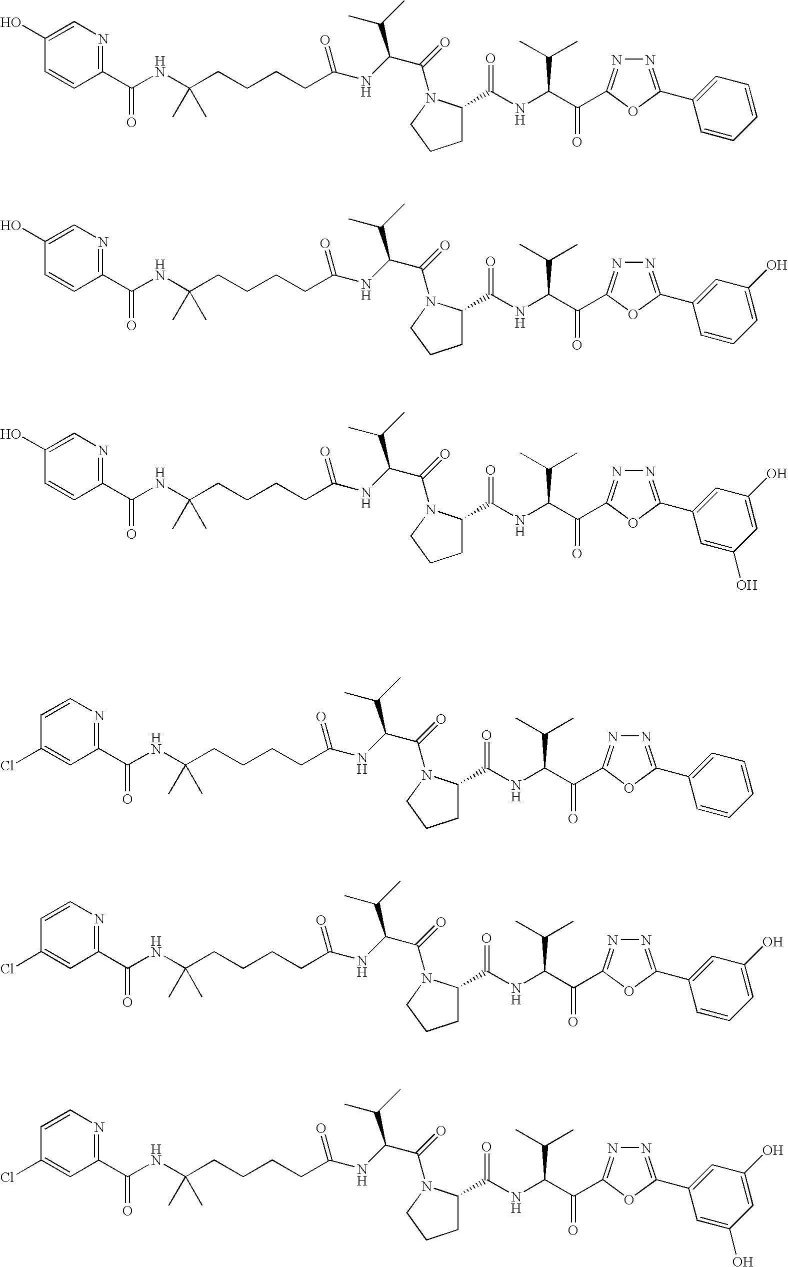

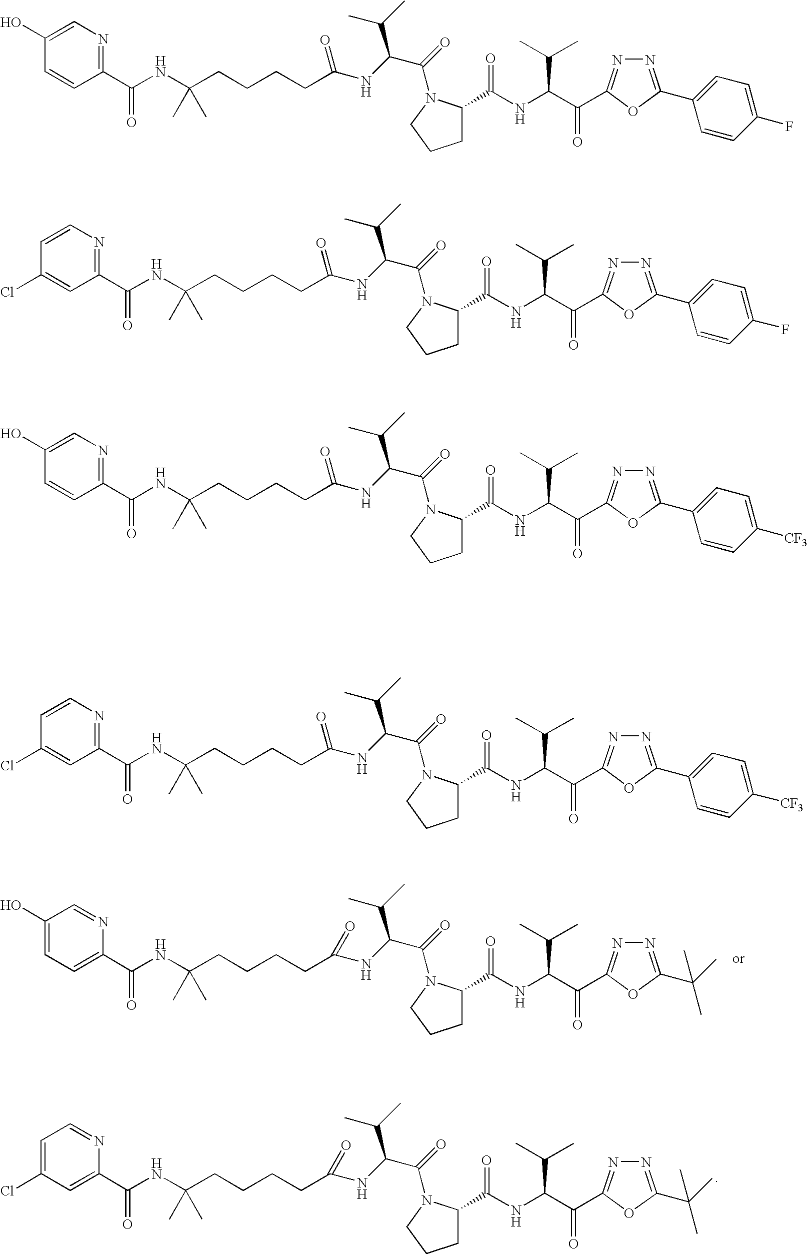

16. A method of treatment for the inhibition of HNE and PR3 which comprises the administration to a subject in need of such inhibition a therapeutically effective amount of a compound of claim 1.

17. The method of treatment of claim 16, wherein the compound of claim 1 is: 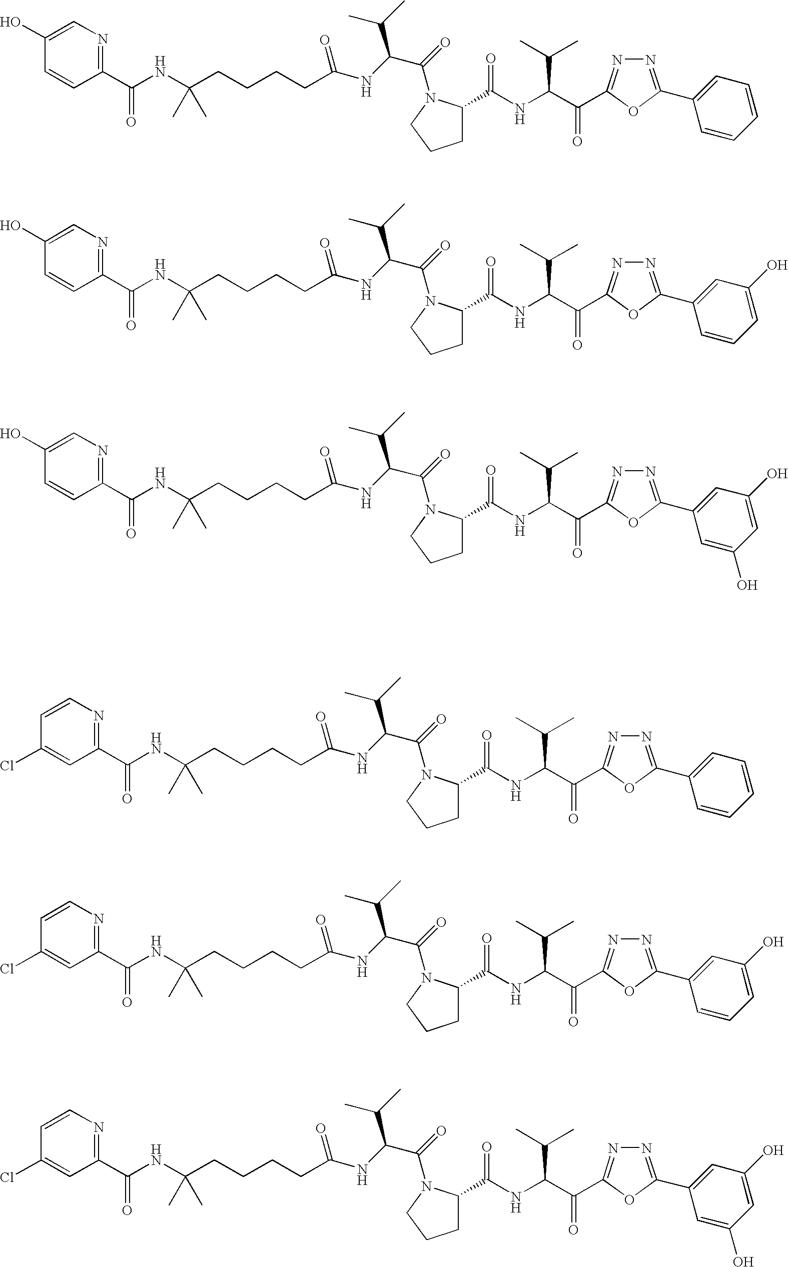 [chemical structure]
